(19) United States
(12) Patent Application Publication
(10) Pub. No.: US 2024/0239902 A1
(43) Pub. Date: Jul. 18, 2024

(54) CO-STIMULATORY MULTISPECIFIC ANTIBODIES

(71) Applicants: Christian-Albrechts-Universität zu Kiel, Kiel (DE); YUMAB GmbH, Braunschweig (DE)

(72) Inventors: Christian KELLNER, Fürstenfeldbruck (DE); Sebastian LUTZ, Kiel (DE); Martin GRAMATZKI, Kiel (DE); Matthias PEIPP, Hamburg (DE); Thomas SCHIRRMANN, Berlin (DE)

(21) Appl. No.: 18/562,279

(22) PCT Filed: May 18, 2022
(86) PCT No.: PCT/EP2022/063381

§ 371 Date: Nov. 17, 2023

(30) Foreign Application Priority Data
May 18, 2021 (EP) 21174284.6

(51) Int. Cl. C07K 16/28; A61K 35/17; A61K 39/00; A61P 35/00; A61P 37/04

(52) U.S. Cl. CPC C07K 16/2851; A61K 35/17; A61K 39/4611; A61K 39/4613; A61K 39/4631; A61K 39/464429; A61P 35/00; A61P 37/04; C07K 16/2809; C07K 16/283; C07K 16/2887; C07K 16/2896; A61K 2039/505; C07K 2317/31; C07K 2317/55; C07K 2317/622

(57) ABSTRACT

The present invention relates to a Fab-scFv fusion protein specifically binding to (i) an antigen being expressed on the surface of a tumor cell or an autoreactive immune cell via the Fab scaffold, and (ii) an antigen being expressed on the surface of a leukocyte, preferably cytotoxic lymphocyte via the scFv fragment

The present invention relates to a multispecific antibody specifically binding to (i) an antigen being expressed on the surface of a tumor cell or an autoreactive immune cell, and (ii) an antigen being expressed on the surface of a leukocyte, preferably cytotoxic lymphocyte.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Since the approval of rituximab in 1997 antibody-based immunotherapy rapidly became the fourth pillar in the treatment of cancer besides surgery, radiation and chemotherapy. However, despite this success story not all patients benefit and relapse of the disease is still a major serious issue [1]. Therefore, further development and optimization of antibody therapy is a major objective in current translational research. The recruitment of effector cells plays an important role for the efficacy of therapeutic antibodies as revealed in murine tumor models and by observations in patients [2-4].

Accordingly, approaches improving this effector function are particular attractive. This mechanism is based on the interaction of the antibody fragment crystallizable (Fc) domain and activating Fcγ-receptors expressed on effector cells, resulting in antibody dependent cellular phagocytosis (ADCP) or antibody dependent cell-mediated cytotoxicity (ADCC). Especially in the treatment of minimal residual disease (MRD) antibody therapy may be promising, since in this situation high effector-to-target cell ratios (E:T ratios) are accessible. However, in patients recruitment of effector cells through therapeutic antibodies is often not sufficient [5]. To overcome this limitation various strategies were developed to optimize effector cell engagement, for example by optimizing the Fc-domain of immunoglobulin G (IgG) antibodies (Fc-engineering) [6], combining monoclonal antibodies (mAbs) with immune stimulatory molecules [7] or attempts using bispecific antibodies [8].

Bispecific antibodies (bsAbs) for effector cell recruitment represent a promising class of therapeutic agents for immunotherapy, in particular in cancer immunotherapy.

These molecules combine two binding moieties of different specificity, the first to target an antigen on tumor cells and the second to trigger an activating receptor on effector cells. Especially natural killer (NK) and T cells can be activated by these agents efficiently, when targeting FcγRIIIa (CD16a), CD3 or γδ T cell receptor respectively [8, 9]. Beside chimeric antigen receptor (CAR) T cells, CD3 bsAbs constitute the most powerful agents for induction of major histocompatibility complex (MHC) independent T cell responses against cancer [10]. Currently, with the bispecific T cell engager (BiTE) blinatumomab, a [CD19×CD3] bispecific single chain fragment variable (bsscFv), one member of this class already has received marketing approval in both the US and the EU in the treatment of relapsed or refractory B cell precursor acute lymphoblastic leukemia and demonstrated promising results [11, 12].

However, there is still an ongoing need for novel bispecific antibodies which are suitable for immunotherapy, in particular in cancer immunotherapy. This need is addressed by the present invention. The strategy of the invention is to stimulate effector cell populations to selectively kill cancer or autoimmune cells and by a co-stimulatory approach to improve the cytotoxic potential of cytotoxic lymphocytes.

Hence, the present invention relates in a first aspect to a multispecific antibody specifically binding to (i) an antigen being expressed on the surface of a tumor cell or an autoreactive immune cell, and (ii) an antigen being expressed on the surface of a leukocyte, preferably a cytotoxic lymphocyte.

The term “multispecific antibody” as used in accordance with the present invention comprises, for example, binding motifs of the at least two different monoclonal antibodies displaying binding specificity to the targets as defined in the above items (i) and (ii). The multispecific antibody may also be extended by a third specificity binding a target on tumor or effector cells. The binding motifs of the at least two different monoclonal antibodies may be comprised in the multispecific antibody in the format of full-length antibodies but also as derivatives or fragments thereof, which still retain the binding specificity to the target, for example an antigen being expressed on the surface of a tumor cell, are comprised in the term “antibody”. Antibody fragments or derivatives comprise, inter alia, Fab or Fab′ fragments, Fd, F(ab′)2, Fv or scFv fragments, single domain VH or V-like domains, such as VhH or V-NAR-domains.

The term “multispecific antibody” also includes embodiments such as chimeric (human constant domain, non-human variable domain), single chain and humanised (human antibody with the exception of non-human CDRs) multispecific antibodies.

Various techniques for the production of antibodies that can be used to prepare multispecific antibodies are well known in the art and described, e.g. in Harlow and Lane (1988) and (1999) and Altshuler et al., 2010, loc. cit. Thus, polyclonal antibodies can be obtained from the blood of an animal following immunisation with an antigen in mixture with additives and adjuvants and monoclonal antibodies can be produced by any technique which provides antibodies produced by continuous cell line cultures. Examples for such techniques are described, e.g. in Harlow E and Lane D, Cold Spring Harbor Laboratory Press, 1988; Harlow E and Lane D, Using Antibodies: A Laboratory Manual, Cold Spring Harbor Laboratory Press, 1999 and include the hybridoma technique originally described by Köhler and Milstein, 1975, the trioma technique, the human B-cell hybridoma technique (see e.g. Kozbor D, 1983, Immunology Today, vol. 4, 7; Li J, et al. 2006, PNAS, vol. 103(10), 3557) and the EBV-hybridoma technique to produce human monoclonal antibodies. Furthermore, recombinant antibodies may be obtained from monoclonal antibodies or can be prepared de novo using various display methods such as phage, ribosomal, mRNA, or cell display. A suitable system for the expression of the recombinant (humanised) antibodies may be selected from, for example, bacteria, yeast, insects, mammalian cell lines or transgenic animals or plants (see, e.g., U.S. Pat. No. 6,080,560; Holliger P, Hudson P J. 2005, Nat Biotechnol., vol. 23(9), 11265). Further, techniques described for the production of single chain antibodies (see, inter alia, U.S. Pat. No. 4,946,778) can be adapted to produce single chain antibodies specific for an epitope of GSK-3. Surface plasmon resonance as employed in the BIAcore system can be used to increase the efficiency of phage antibodies.

In accordance with the present invention the antibody is a multispecific antibody having a multi-chain or single-chain format. Multi-chain or single-chain antibody formats are, for example, minibodies, diabodies, bibodies, tribodies or triplebodies, tetrabodies or chemically conjugated Fab′-multimers (see, for example, Harlow and Lane “Antibodies, A Laboratory Manual”, Cold Spring Harbor Laboratory Press, 1988; Harlow and Lane “Using Antibodies: A Laboratory Manual” Cold Spring Harbor Laboratory Press, 1999; Altshuler E P, Serebryanaya D V, Katrukha A G. 2010, Biochemistry (Mosc)., vol. 75(13), 1584; Holliger P, Hudson P J. 2005, Nat Biotechnol., vol. 23(9), 1126). Among the formats the bibody format is preferred since bibodies are illustrated by the examples. The bibody, a Fab-scFv fusion protein, is created by adding a scFv fragment to the C-terminus of Fab scaffold. In this class of multispecific antibodies the bispecific fragment utilizes the natural in vivo heterodimerization of the Fd fragment (the HC regions of Fab fragment) and light chain. The heterodimerization scaffold can be further incorporated with additional functions, such as scFvs, scaffold proteins, cytokines, etc. to form bivalent, bispecific molecules or trivalent, bi- or tri-specific molecules. The bibody molecules, Fab-L-scFv and Fab-H-scFv, are bispecific and bivalent. It has been shown that this format can retain the bispecific binding, a low tendency to aggregate and stable in physiological conditions. The multi-chain formats in particular comprise bispecific antibodies that can simultaneously bind to two different types of antigens. Non-limiting examples of bispecific antibodies formats are Biclonics (bispecific, full length human IgG antibodies), DART (Dual-affinity Re-targeting Antibody) and BiTE (consisting of two single-chain variable fragments (scFvs) of different antibodies) molecules (Kontermann and Brinkmann (2015), Drug Discovery Today, 20(7):838-847). Further bispecific antibodies formats will be discussed herein below.

The term “multispecific antibody” as used herein refers to an antibody that possesses at least two different binding domains and is thus capable of specifically binding to two different epitopes. In case the antibody possesses two binding domains it may be referred to as a “bispecific binding antibody”. In accordance with the invention the first epitope is part of an antigen being expressed on the surface of a tumor cell or an autoreactive immune cell, and the second epitope is part an antigen being expressed on the surface of a leukocyte, preferably a cytotoxic lymphocyte.

An antigen as used herein refers to a molecule or molecular structure being present on the outside of a cell, that can be specifically bound by the multispecific binding antibody of the invention. The antigen comprises an epitope (also called antigenic determinant), which is the part of an antigen that is recognized by the multispecific binding antibody of the invention.

Tumor cells are aberrant cells that differ from normal body cells in many ways. Normal cells become tumor cells when a series of mutations leads the cell to continue to grow and divide out of control. Also unlike normal cells, tumor cell cells may have the ability to invade nearby tissues and/or spread to distant regions of the body. The series of mutations often results in the expression of an antigen on the surface of a tumor cell that is not expressed on normal cells. In addition, tumor cells express various antigens found also on heathy tissues. However, also such antigens can be used as target structures, given that they have a limited expression pattern and/or their expression is restricted to certain tissues or cell types in heathy tissues. Such antigens are referred to herein as antigens being expressed on the surface of a tumor cell. The antigen is preferably not expressed on normal cells. The “bispecific binding antibody” may thus specifically or at least highly preferentially bind to tumor cells.

The tumor as referred to herein may be malignant or benign tumor. The tumor is preferably a malignant tumor, which is also referred to herein as cancer.

Also autoreactive immune cells are aberrant cells. They differ from normal immune cells in that they are not directed against foreign antigens but are directed to an antigen that can be found in the body of the subject producing these cells. Thereby these cells trigger immune responses of an organism against its own healthy cells, tissues and other body normal constituents. Autoreactive immune cells may therefore turn to be harmful and cause autoimmune diseases, such as multiple sclerosis, arthritis or lupus erythematosus.

Leukocytes are also called white blood cells. Leukocytes can be divided into the five main types: neutrophils, eosinophils, basophils, lymphocytes, and monocytes. The leukocyte is preferably a cytotoxic lymphocyte. The cytotoxic lymphocyte is preferably a cytotoxic T-cell, a natural killer T (NKT) cell or a natural killer cell (NK cell). NKT cells are a heterogeneous group of T cells that share properties of both T cells and NK cells. In this connection the term “cytotoxic” refers to the capability of the cells to specifically kill cells, for example by binding to an antigen being expressed on the cell and/or by mediating antibody-dependent cell-mediated cytotoxicity (ADCC). For instance, in the case of a tumor specific cytotoxic T-cell the T-cell receptor on the surface of the cytotoxic T-cell is specific for a tumor antigen. The TCR binds to the antigen and the cytotoxic T-cell destroys the cell.

Hence, the multispecific antibody of the invention on the one hand binds to aberrant cells (tumor cells or autoimmune cells) and on the other hand binds to cytotoxic leukocytes (e.g. NK cell, NTK cells and/or cytotoxic T-cells). In this context the multispecific antibody stimulates the immune system (immune modulator function) and sensitizes the aberrant cell to depletion by cytotoxic leukocytes. The immune modulator function is highly selective for the aberrant cells and capable of stimulating cytotoxic leukocytes, so that they kill the aberrant cells. However, this function alone is not sufficient to effectively kill the aberrant cells. It was surprisingly found that the immune modulator function as a co-activator within the multispecific antibody of the invention is capable of significantly enhancing the cytotoxic effect as mediated by a second antibody or antibody-derivative (immune activator), which engages an independent antigen on the same cytotoxic leukocyte and a different antigen on the tumor cell. The cytotoxic effect was increased by a factor of about 10 in the case of CD20×NKG2D antibodies; see Examples 6 and 7. The essentially same was found for HER2×NKG2D, CD138×NKG2D and CD319CS1×NKG2D antibodies; see Examples 8 and 9. Examples 6 to 9 evidence the broad applicability of the increase of the cytotoxic effect, noting that all tested multispecific antibodies were produced in the format of a Fab-scFv fusion protein. The Fab-scFv fusion protein is believed to be particular advantageous in order to achieve the increase of the cytotoxic effect.

Bispecific antibodies such as blinatumomab are characterized by strong immune activator function and trigger immune responses alone by engaging CD3-positive lymphocytes resulting in robust activation. This strong own activity as single agent and a lack of expression of CD3 by NK cells precludes its use as an immune modulator for NK cell co-activation and makes it inappropriate for the use as an immune modulator for T cells. Also, fusion of an NKG2D ligand to an anti-CD20-single chain antibody fragment resulted in a considerable high own cytotoxic activity when applied alone, being therefore rather an immune activator than an immune modulator. In this context, an immune activator is seen as a molecule that as a single agent triggers immune cells efficiently also without co-stimulation (e.g. induces cytotoxicity when applied alone in a standardized 4 h chromium release experiment), whereas an immune modulator may exert a weak single-agent activity, but modulates effects induced by an immune activator. Preferably, the immune modulator functions as an enhancer that sensitizes target cells to the effects triggered by an immune activator (e.g. potentiates its cytotoxic activity), while being almost ineffective in the absence of an immune activator.

In accordance with a preferred embodiment of the first aspect of the invention the antigen of (ii) is expressed on natural killer (NK) cells, natural killer T (NKT) cells and/or cytotoxic thymocytes (T cells), and/or genetically engineered cells thereof.

In this connection it is preferred that the antigen of (ii) is expressed on natural killer (NK) cells and cytotoxic thymocytes (T cells) since this leads to a cytotoxic effect mediated by both cell types, natural killer (NK) cells and cytotoxic thymocytes (T cells). Examples of such antigens will be provided herein below.

Means and methods for producing genetically engineered cytotoxic lymphocytes are known in the art, for example, from Bonini et al., Biol Blood Marrow Transplant. 2011 January; 17(1 Suppl): S15-S20. For instance, cytotoxic specific T lymphocytes (CTLs) may be isolated from a subject, expanded ex-vivo and then primed to recognize a particular antigen or are genetically modified to express a particular TCR or a CAR recognizing a target antigen. Such modified T cells may then be used treat the subject by an autologous T lymphocyte therapy.

In accordance with a further preferred embodiment of the first aspect of the invention the antigen of (ii) is selected from the group consisting of NKG2D, CD137, NKp30, NKp46, NKp44, 2B4, DNAM-1, CD2, CD4, CD8 and CD28, wherein the antigen is preferably NKG2D.

The multispecific antibody may include binding domains to more than one of these antigens. The multispecific antibody may accordingly bind to two or more antigens selected from the group consisting of NKG2D, CD137, NKp30, NKp46, NKp44, 2B4, DNAM-1, CD2, CD4, CD8 and CD28, wherein the two or more antigens preferably comprise NKG2D.

NKG2D is a transmembrane protein belonging to the NKG2 family of C-type lectin-like receptors. NKG2D plays a key role in immune surveillance of tumors and pathogens [13, 14]. In humans, NKG2D is expressed by NK cells and cytotoxic thymocytes and recognizes “induced-self proteins”, which are frequently expressed at the cell surface after viral infection or malignant transformation [15, 16]. Human NKG2D ligands include MHC class I-related chain (MIC) A and B as well as UL16-binding proteins (ULBP) 1-6. Recognition of these danger signaling antigens results in cell activation through an intracellular activation pathway via the NKG2D-associated adapter protein DNAX-activating protein of 10 kDa (DAP10) [17]. In NK cells, this signal promotes natural cytotoxicity [18]. In contrast to NK cells, the role of NKG2D in T cells is more complex. It is expressed by CD8⁺ αβ T cells, γδ T cells, NKT cells as well as by subsets of CD4 T cells in humans. Previous studies showed that co-stimulation of NKG2D regulates priming, proliferation and function of cytotoxic T cells [24, 25]. However, after prolonged stimulation with IL-2 or IL-15, T cells were also able to kill target cells TCR-independent via NKG2D activation [26, 27]. It was shown that the expression of NKG2D ligands on tumor cells leads to a T cell mediated adaptive immune response in a syngeneic murine tumor model [28]. During cancer progression many tumors escape this immune surveillance mechanism through downregulation or proteolytic shedding of NKG2D ligands (Salih et al., J Immunol. 2002 Oct. 15; 169(8):4098-102). Therefore, different strategies had been pursued to restore NKG2D-mediated recognition of malignant cells. It was, for example, shown that bispecific immunoligands engaging NKG2D trigger NK cell cytotoxicity and synergistically enhance NK cell-mediated ADCC by therapeutic antibodies [19, 20]. An analogously constructed molecule targeting multiple myeloma (MM) cells also showed promising results in vitro and in vivo in a xenograft mouse model [21]. Furthermore, a bsAb with specificities for NKG2D and CS-1 was shown to exert therapeutic effects in preclinical models of MM recently [22]. Another recent study used anti-MICA and anti-MICB antibodies to inhibit shedding of these ligands, resulting in enhanced NK cell cytotoxicity trough NKG2D and additional FcγRIIIa activation [23]. As NKG2D is expressed on NK cells as well as on T cells, it is one example of an antigen being expressed on the surface of a cytotoxic leukocyte, and in particular on both NK cells and T cells.

CD137 (ILA/4-1BB) is a member of the tumor necrosis factor receptor family, expressed on activated T lymphocytes and NK cells.

NKp30 (CD337) is a stimulatory receptor on human NK cells implicated in tumor immunity, and is capable of promoting or terminating dendritic cell maturation.

NKp46 is a major NK cell-activating receptor that is involved in the elimination of target cells being killed by NK cells.

NKp44 (CD336) is a member of Natural Cytotoxicity Receptors (NCRs). It is an activating receptor playing a crucial role in most functions exerted by activated NK cells and also by other NKp44+ immune cells.

2B4 (CD244) is a natural killer cell receptor mediating non-major histocompatibility complex (MHC) restricted killing.

DNAM-1 (CD226) is a ˜65 kDa glycoprotein being expressed on the surface of natural killer cells, platelets, monocytes and a subset of T cells. It is a member of the immunoglobulin superfamily containing 2 Ig-like domains of the V-set.

CD2 is a cell adhesion molecule found on the surface of T cells and natural killer (NK) cells. It has known as T-cell surface antigen T11/Leu-5, LFA-2, LFA-3 receptor, erythrocyte receptor and rosette receptor.

CD4 is a glycoprotein found on the surface of immune cells, such as T helper cells, monocytes, macrophages, and dendritic cells. CD4 is a co-receptor of the T cell receptor (TCR) and assists the latter in communicating with antigen-presenting cells.

CD8 is a transmembrane glycoprotein that serves as a co-receptor for the T-cell receptor (TCR). Together with the TCR, the CD8 co-receptor plays a role in T cell signaling and is involved in cytotoxic T-cell antigen interactions.

CD28 is expressed on T cells that provide co-stimulatory signals required for T cell activation and survival. T cell stimulation through CD28 in addition to the T-cell receptor (TCR) can provide a potent signal for the production of various interleukins (IL-6 in particular).

In accordance with another preferred embodiment of the first aspect of the invention the antigen of (i) is selected from the group consisting of CD20, CD19, CD22, CD37, CD38, CD7, CD33, CD44, CD54, CD64, CD75s, CD79b, CD96, CD138, CD123, CD317, CD319, BCMA, FCRL5, EGFR, HER2, EpCAM CEA, GD2 and Claudin 6/18.2 wherein the antigen is preferably CD20.

The multispecific antibody may include binding domains to more than one of these antigens. The multispecific antibody may accordingly bind to two or more antigens selected from the group consisting of CD20, CD19, CD22, CD37, CD38, CD7, CD33, CD44, CD54, CD64, CD75s, CD79b, CD96, CD123, CD138, CD317, CD319, BCMA, FCRL5, EGFR, HER2, EpCAM CEA and Claudin 6/18, wherein the two or more antigens preferably comprise CD20.

CD20 B-lymphocyte antigen CD20 or CD20 is expressed on the surface of all B-cells beginning at the pro-B phase (CD45R+, CD117+) and progressively increasing in concentration until maturity. CD20 has been found on B-cell lymphomas, hairy cell leukemia, chronic lymphocytic leukemia (CLL), B-cell acute lymphoblastic leukemia (ALL) and melanoma cancer stem cells.

CD19 is a is a transmembrane protein being expressed in B cells. Since CD19 is a marker of B cells, the protein has been used to diagnose and target cancers that arise from this type of cell, notably B cell lymphomas, acute lymphoblastic leukemia (ALL), and chronic lymphocytic leukemia (CLL).

CD22 is a molecule belonging to the SIGLEC family of lectins and is found on the surface of mature B cells and to a lesser extent on some immature B cells. Also CD22 has been used to diagnose and target cancers that arise from B cells, such as acute lymphoblastic leukemia (ALL).

CD37 is a member of the transmembrane 4 superfamily. The expression of CD37 is restricted to cells of the immune system, with highest abundance on mature B cells, and lower expression is found on T cells and myeloid cells. In cancer, CD37 is highly expressed on malignant B cells in a variety of B-cell lymphomas and leukemias, including Non-Hodgkin lymphoma (NHL) and CLL.

CD38 is a glycoprotein found on the surface of many immune cells (white blood cells), including CD4+, CD8+, B lymphocytes and natural killer cells. CD38 is also expressed in various hematological malignancies including NHL, MM, CLL and ALL.

CD7 encodes a transmembrane protein which is a member of the immunoglobulin superfamily. CD7 is found on thymocytes and mature T cells. CD7 is expressed by T lineage leukemias and lymphomas and is a leukemic prognostic marker.

CD33 is a transmembrane receptor being expressed on cells of myeloid lineage. It is a target used for treatment of patients with acute myeloid leukemia.

CD44 is a cell-surface glycoprotein being involved in cell-cell interactions, cell adhesion and migration. CD44 is expressed in a large number of mammalian cell types. Variations in CD44 are reported as cell surface markers for some breast and prostate cancer stem cells.

CD54 is a cell surface glycoprotein which is typically expressed on endothelial cells and cells of the immune system. CD54 has an important role in ocular allergies recruiting pro-inflammatory lymphocytes and mast cells promoting a type I hypersensitivity reaction.

CD64 is a type of integral membrane glycoprotein known as an Fc receptor that binds monomeric IgG-type antibodies with high affinity. CD64 is found on macrophages and monocytes. Neutrophil CD64 expression is increased in inflammatory autoimmune diseases.

CD75s is an alpha-2,6-sialylated carbohydrate epitope being expressed by mature B cells (especially germinal centre B cells), red blood cells and some epithelial cells. CD75s has been identified as a promising target for immunotherapy of mature B cell malignancies.

CD79b is the B-cell antigen receptor complex-associated protein beta chain. Diseases associated with CD79b include agammaglobulinemia 6, autosomal recessive and agammaglobulinemia, Non-Bruton type.

CD96 is a transmembrane glycoprotein that has three extracellular immunoglobulin-like domains and is expressed by resting NK cells. CD96 has been reported to correlate with immune profile and clinical outcome of glioma.

CD123 is a molecule found on cells which helps transmit the signal of interleukin-3, a soluble cytokine important in the immune system, such as pluripotent progenitor cells of hematopoietic cells. CD123 is expressed across acute myeloid leukemia (AML) subtypes, including leukemic stem cells.

CD138 (or syndecan 1) is a protein which in humans is encoded by the SDC1 gene. The protein is a transmembrane (type I) heparan sulfate proteoglycan. CD138 functions as an integral membrane protein and participates in cell proliferation, cell migration and cell-matrix interactions via its receptor for extracellular matrix proteins. CD138 is a sponge for growth factors and chemokines, with binding largely via heparan sulfate chains.

CD317 is a lipid raft associated protein being expressed in mature B cells, plasma cells and plasmacytoid dendritic cells, and in many other cells. It is only expressed as a response to stimuli from the IFN pathway. Several reports have described the expression of CD137 in various types of malignancies, including lung cancer, leukemia, and lymphoma.

CD319 (also known as CS1 (CD2 subset-1), CRACC and SLAMF7) is a single-pass type I transmembrane glycoprotein, expressed on NK cells, subsets of mature dendritic cells, activated B cells, and cytotoxic lymphocytes, but not in promyelocytic, B or T cell lines. CD319 is a robust marker of normal plasma cells and malignant plasma cells in multiple myeloma.

BCMA (B-cell maturation antigen) is a cell surface receptor of the TNF receptor superfamily which recognizes B-cell activating factor (BAFF). BCMA is implicated in leukemia, lymphomas, and multiple myeloma.

FCRL5 (Fc receptor-like protein 5, also known as CD307) is a receptor that recognizes intact IgG, possibly enabling B cells to sense Ig quality. Diseases associated with FCRL5 include hairy cell leukemia and lymphoma.

EGFR (epidermal growth factor receptor) is a transmembrane protein that is a receptor for members of the epidermal growth factor family (EGF family) of extracellular protein ligands. In many cancer types, mutations affecting EGFR expression or activity could result in cancer.

HER2 (Receptor tyrosine-protein kinase erbB-2, also known as CD340) is a receptor having an important role in normal cell growth and differentiation. HER 2 over-expression is known to occur, for example, in breast, ovarian, stomach, adenocarcinoma of the lung, and uterine cancer.

EpCAM (epithelial cell adhesion molecule) is a transmembrane glycoprotein mediating Ca2+-independent homotypic cell-cell adhesion in epithelia. EpCAM is overexpressed in many carcinomas and in cancer stem cells, making EpCAM an attractive target for immunotherapy.

CEA (Carcinoembryonic antigen) describes a set of highly related glycoproteins involved in cell adhesion. CEA is normally produced in gastrointestinal tissue during fetal development, but the production stops before birth. In adults, CEA is primarily expressed in cells of (malignant and benign) tumors.

GD2 is a disialogangliside with limited expression in healthy tissues. In certain tumors, GD2 is extensively expressed and has been associated with cancer development. The antigen is a target in the treatment of neuroblastoma.

CLDNs (claudins) refers to the members of a family of proteins which, along with occludin, are the most important components of the tight junctions (zonulae occludentes). Altered expression of several claudin proteins, in particular claudin-1, -3, -4 and -7, has been linked to the development of various cancers. In addition, CLDN6 and CLDN18.2 are attractive target for immunotherapy.

In accordance with a preferred embodiment of the first aspect of the invention the antigen of (ii) is NKG2D and the antibody competes with the natural ligand ULPB2 for binding to NKG2D.

As mentioned above NKG2D is expressed on NK cell and cytotoxic T cells and, thus, the most preferred antigen of (ii) in accordance with the claimed invention. NKG2D is also expressed on CD4⁻ NKT cells, whereas most CD4⁺ NKT cells lack this receptor (Kuylenstierna el al., Eur J Immunol. 2011 July; 41(7): 1913-1923). The NKT cells as referred to herein are therefore preferably CD4⁻ NKT cells.

By upregulating “stress-induced ligands” damaged or transformed cells can be recognized by immune cells and cleared. The human genome encodes eight functional “stress-induced ligands”: MICA, MICB, and ULBP1-6. All of them are recognized by a single receptor, NKG2D.

The natural ligands ULBP3, UBP6, MICA and presumably the other known ligands of NKG2D share a binding region on the NKG2D receptor, which is a preferred binding site for antibodies to trigger NK/T cell activation. The fine epitope and affinity may significantly impact the strength of activation/co-stimulation.

In accordance with a yet further preferred embodiment of the first aspect of the invention the multispecific antibody comprises Fab, scFv, Fv, VHH, and/or dAb scFv fragments as components, and preferably is an IgG-scFv or a Fab-scFv fusion protein.

While the particular format of the multispecific antibody of the invention is not particularly limited, the multispecific antibody in accordance with this preferred embodiment comprises as Fab, scFv, Fv, VHH, or dAb as components.

Fab (antigen-binding fragment), scFv (single-chain fragment variable), Fv (fragment variable), VHH (variable domain of a heavy only antibody) and dAb (domain antibody) are well-known fragments of a full (or complete) antibody. A full (or complete) antibody consists of each two copies of the entire light and heavy immunoglobulin chains. Among this list of antibody fragments a scFv fragment is particularly preferred as being comprised in the multispecific antibody of the invention.

The distinguishing properties of antibody fragments as compared to full-length antibodies are, for example, a smaller size, monovalent antigen binding, lack of FcR binding, general lack of complex glycosylation and/or robust biophysical properties.

The format of the multispecific antibody of the invention is preferably an IgG-scFv or a Fab-scFv fusion protein. In the first case an IgG (i.e. full IgG antibody) is fused to a scFv fragment and in the second case a Fab fragment is fused to a scFv fragment.

In the preferred cases where the multispecific antibody of the invention comprises a scFv fragment, said scFv fragment is preferably fused via a peptide-linker, more preferably via a GS-linker to the C-terminus of a Fab, IgG, or a Fc scaffold.

A Fc scaffold comprises or consists of the constant region of an antibody. A peptide-linker is a short amino acid sequence, preferably in the range of 5 to 50 amino acids. A GS-linker consists only of glycine and serine amino acids.

In this connection it is to be understood that the Fc scaffold does not comprise an antigen binding site but is a further component of the multispecific antibody. The Fc scaffold can, for example, increase the in vivo serum stability and retention time of the multispecific antibody.

In accordance with a more preferred embodiment of the first aspect of the invention the Fab scaffold specifically binds to the antigen of (i) and the scFv fragment specifically binds to the antigen of (ii).

This particular Fab-scFv format of a multispecific antibody of the invention is illustrated in the examples of the application as filed and, thus, particularly preferred. The Fab-scFv format with an intermediate molecular mass of about 75 kDa may—in contrast to the tandem scFv format—not be eliminated by renal clearance thereby prolonging its in vivo half-life. Compared to IgG-like formats the smaller size displays favorable characteristics in mediating synapse formation between target and effector cell. Obviating the use of multiple scFv fragments such Fab-scFv molecules show less tendency to form multimers or aggregates.

In case even further prolonged in vivo half-life is desired the Fab-scFv format can be equipped in addition with an Fc domain. Such molecules with a molecular mass of about 125 kDa are still smaller than regular IgG antibodies and may therefore demonstrate favorable characteristics in terms of tissue penetration.

In accordance with a preferred embodiment of the first aspect of the invention the multispecific antibody comprises in case of (i) the six CDRs of SEQ ID NOs 22 to 26 and the CDR2 VL ATS; and/or in case of (ii) the six CDRs of SEQ ID NOs 1 to 5 and the CDR2 VL GNN or SEQ ID NOs 6 to 10 and the CDR2 VL GKN or SEQ ID NOs 11 to 15 and the CDR2 VL GKN.

It can be taken from the examples herein below that the inventors produced 38 anti-NKG2D antibodies (clones 1 to 38) and processed 36 of them into Fab-scFv fusion proteins (bibodies), wherein the Fab fragment is directed against CD20 and the scFv fragment is directed against NKG2D.

While the 36 Fab-scFv fusion proteins are all capable of inducing NK cell activation, among the 36 Fab-scFv fusion proteins the three clones with the anti-NKG2D antibodies 3, 32 and 35 as referred to in the examples were capable of inducing the most potent NK cell activation. Among clones 3, 32 and 35 clones 3 and 32 induced and even better NK cell activation than clone 35 and clone 3 provides the additional advantage of binding to human and murine NKG2D (see Example 10). The Fab-scFv fusion proteins with the anti-NKG2D antibodies 3 and 32 were capable of potentially lysing lymphoma cells. The human and murine NKG2D is advantageous for pre-clinical tests in a mouse model. Among clones 3, 32 and 35, clones 3 and 32 are therefore preferred and clone 3 is most preferred.

SEQ ID NOs 1 to 5 and the CDR2 VL GNN. or SEQ ID NOs 6 to 10 and the CDR2 VL GKN, or SEQ ID NOs 11 to 15 and the CDR2 VL GKN are the sets of six CDR sequences of three novel anti-NKG2D antibodies clones 3, 32 and 35, respectively. Among SEQ ID NOs 1 to 5 and the CDR2 VL GNN, or SEQ ID NOs 6 to 10 and the CDR2 VL GKN, or SEQ ID NOs 11 to 15 and the CDR2 VL GKN, SEQ ID NOs 1 to 5 and the CDR2 VL GNN or SEQ ID NOs 6 to 10 and the CDR2 VL GKN are preferred and SEQ ID NOs 1 to 5 and the CDR2 VL GNN are most preferred.

The six CDRs of SEQ ID NOs 22 to 26 and CDR VL ATS are the six CDRs of the commercially available CD20 antibody rituximab. Rituximab is used in the art for the treatment of autoimmune diseases and types of cancer. It is, for example, used for non-Hodgkin lymphoma, chronic lymphocytic leukemia, rheumatoid arthritis, granulomatosis with polyangiitis, idiopathic thrombocytopenic purpura, pemphigus vulgaris, myasthenia gravis and Epstein-Barr virus-positive mucocutaneous ulcers.

While rituximab (in Fab format) has been used as an example of an antibody that specifically binds to an antigen being expressed on the surface of a tumor cell or an autoreactive immune cell the discussed advantages of clones 3, 32 and 35 are not limited to enhancing the efficacy of rituximab in the format of a multispecific antibody. It is at least highly plausible that clones 3, 32 and 35 can enhance the efficacy of any therapeutic antibody that specifically binds to an antigen being expressed on the surface of a tumor cell or an autoreactive immune cell.

For this reason, within the above preferred embodiment of the first aspect the multispecific antibody preferably comprises (i) an antibody, preferably a scFv fragment comprising the six CDRs of SEQ ID NOs 1 to 5 and the CDR2 VL GNN. or SEQ ID NOs 6 to 10 and the CDR2 VL GKN, or SEQ ID NOs 11 to 15 and the CDR2 VL GKN; and (ii) an antibody, preferably a Fab fragment that specifically binds to an antigen being expressed on the surface of a tumor cell or an autoreactive immune cell.

The present invention also relates to an anti-NKG2D antibody comprising the six CDRs of SEQ ID NOs 1 to 5 and the CDR2 VL GNN. or SEQ ID NOs 6 to 10 and the CDR2 VL GKN, or SEQ ID NOs 11 to 15 and the CDR2 VL GKN.

The above definitions and preferred embodiments as disclosed in connection with the first aspect apply mutatis mutandis to the anti-NKG2D antibody of the invention.

In accordance with another preferred embodiment of the first aspect of the invention, the multispecific antibody comprises in case of (i) the variable heavy and light chain regions of SEQ ID NOs 27 and 28; and/or in case of (ii) the variable heavy and light chain regions of SEQ ID NOs 16 and 17 or SEQ ID NOs 18 and 19 or SEQ ID NOs 20 and 21.

The variable heavy and light chain regions of SEQ ID NOs 16 and 17, SEQ ID NOs 18 and 19 and SEQ ID NOs 20 and 21 are the heavy and light chain regions of the discussed clones 3, 32 and 35, respectively. Also described herein is multispecific antibody that comprises (i) the variable heavy and light chain regions being at with increased preference at least 90%, at least 95%, at least 98% and at least 99% identical to SEQ ID NOs 16 and 17 or SEQ ID NOs 18 and 19 or SEQ ID NOs 20 and 21; and/or (ii) the variable heavy and light chain regions of SEQ ID NOs 27 and 28. In this connection it is preferred that such a multispecific antibody comprises (i) the six CDRs of SEQ ID NOs 1 to 5 and the CDR2 VL GNN, or SEQ ID NOs 6 to 10 and the CDR2 VL GKN, or SEQ ID NOs 11 to 15 and the CDR2 VL GKN; and/or (ii) the six CDRs of SEQ ID NOs 22 to 26 and the CDR VL ATS with no changes.

In accordance with the present invention, the term “percent (%) sequence identity” describes the number of matches (“hits”) of identical nucleotides/amino acids of two or more aligned nucleic acid or amino acid sequences as compared to the number of nucleotides or amino acid residues making up the overall length of the template nucleic acid or amino acid sequences. In other terms, using an alignment for two or more sequences or subsequences the percentage of amino acid residues or nucleotides that are the same (e.g. 90% or 95% identity) may be determined, when the (sub)sequences are compared and aligned for maximum correspondence over a window of comparison, or over a designated region as measured using a sequence comparison algorithm as known in the art, or when manually aligned and visually inspected. This definition also applies to the complement of any sequence to be aligned.

Nucleotide and amino acid sequence analysis and alignment in connection with the present invention are preferably carried out using the NCBI BLAST algorithm (Stephen F. Altschul, Thomas L. Madden, Alejandro A. Schäffer, Jinghui Zhang, Zheng Zhang, Webb Miller, and David J. Lipman (1997), Nucleic Acids Res. 25:3389-3402). BLAST can be used for nucleotide sequences (nucleotide BLAST) and amino acid sequences (protein BLAST). The skilled person is aware of additional suitable programs to align nucleic acid sequences.

The variable heavy and light chain regions of SEQ ID NOs 27 and 28 are the variable heavy and light chain regions of rituximab.

Within the above preferred embodiment of the first aspect the multispecific antibody preferably comprises (i) an antibody, preferably an scFv fragment comprising the variable heavy and light chain regions of SEQ ID NOs 16 and 17 or SEQ ID NOs 18 and 19 or SEQ ID NOs 20 and 21; and (ii) an antibody, preferably a Fab fragment that specifically binds to an antigen being expressed on the surface of a tumor cell or an autoreactive immune cell.

For the reasons explained above, the present invention also relates to anti-NKG2D antibody comprising the variable heavy and light chain regions of SEQ ID NOs 16 and 17 or SEQ ID NOs 18 and 19 or SEQ ID NOs 20 and 21. In the scFv fragment SEQ ID NOs 16 and 17 or SEQ ID NOs 18 and 19 or SEQ ID NOs 20 and 21 are preferably linked by a peptide linker and more preferably via the linker of SEQ ID NO: 29.

The present invention relates in a second aspect to a nucleic acid sequence or a set of nucleic acid sequences encoding the multispecific antibody of the invention.

The term “nucleic acid molecule” in accordance with the present invention includes DNA, such as cDNA or double or single stranded genomic DNA and RNA. In this regard, “DNA” (deoxyribonucleic acid) means any chain or sequence of the chemical building blocks adenine (A), guanine (G), cytosine (C) and thymine (T), called nucleotide bases, that are linked together on a deoxyribose sugar backbone. DNA can have one strand of nucleotide bases, or two complimentary strands which may form a double helix structure. It further includes RNA. “RNA” (ribonucleic acid) means any chain or sequence of the chemical building blocks adenine (A), guanine (G), cytosine (C) and uracil (U), called nucleotide bases, that are linked together on a ribose sugar backbone. RNA typically has one strand of nucleotide bases, such as mRNA. Included are also single- and double-stranded hybrids molecules, i.e., DNA-DNA, DNA-RNA and RNA-RNA. The nucleic acid molecule may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Nucleic acid molecules, in the following also referred as polynucleotides, may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Further included are nucleic acid mimicking molecules known in the art such as synthetic or semi-synthetic derivatives of DNA or RNA and mixed polymers. Such nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA), peptide nucleic acid (PNA) and locked nucleic acid (LNA) (see Braasch and Corey, Chem Biol 2001, 8: 1). LNA is an RNA derivative in which the ribose ring is constrained by a methylene linkage between the 2′-oxygen and the 4′-carbon. Also included are nucleic acids containing modified bases, for example thio-uracil, thio-guanine and fluoro-uracil. A nucleic acid molecule typically carries genetic information, including the information used by cellular machinery to make proteins and/or polypeptides. The nucleic acid molecule of the invention may comprise promoters, enhancers, response elements, signal sequences, polyadenylation sequences, introns, 5′- and 3′-non-coding regions, and the like.

The nucleic acid molecule according to the invention encodes the multispecific antibody of the invention. The multispecific antibody of the invention may also be encoded by a set of nucleic acid molecules, preferably by a set of two nucleic acid molecules. This is because an antibody (a full-length antibody, scFv or Fab) comprises heavy and light chain sequences which, for example, upon expression in a cell, self-assemble into an antibody. The heavy and light chain sequences can be encoded by a set of different nucleic acid molecules, preferably by two nucleic acid molecules.

The present invention relates in a third aspect to a vector or a set of vectors encoding the multispecific antibody of the invention in expressible from.

The term “vector” in accordance with the invention means preferably a plasmid, cosmid, virus, bacteriophage or another vector used e.g. conventionally in genetic engineering which encoding the multispecific antibody of the invention in expressible form. For the same reasons as discussed in connection with the set of nucleic acid molecules of the invention, the multispecific antibody of the invention may also be encoded by a set of vectors, preferably by a set of two vectors.

The nucleic acid molecule(s) encoding the multispecific antibody of the invention may, for example, be inserted into several commercially available vectors. Non-limiting examples include prokaryotic plasmid vectors, such as of the pUC-series, pBluescript (Stratagene), the pET-series of expression vectors (Novagen) or pCRTOPO (Invitrogen) and vectors compatible with an expression in mammalian cells like pREP (Invitrogen), pSec Tag2 (Invitrogen), pcDNA3 (Invitrogen), pCEP4 (Invitrogen), pMC1neo (Stratagene), pXT1 (Stratagene), pSG5 (Stratagene), EBO-pSV2neo, pBPV-1, pdBPVMMTneo, pRSVgpt, pRSVneo, pSV2-dhfr, pIZD35, pLXIN, pSIR (Clontech), pIRES-EGFP (Clontech), pEAK-10 (Edge Biosystems) pTriEx-Hygro (Novagen) and pCINeo (Promega). Examples for plasmid vectors suitable for Pichia pastoris comprise e.g. the plasmids pAO815, pPIC9K and pPIC3.5K (all Invitrogen).

The nucleic acid molecules inserted into the vector can e.g. be synthesized by standard methods, or isolated from natural sources. Ligation of the coding sequences to transcriptional regulatory elements and/or to other amino acid encoding sequences can also be carried out using established methods. Transcriptional regulatory elements (parts of an expression cassette) ensuring expression in prokaryotes or eukaryotic cells are well known to those skilled in the art. These elements comprise regulatory sequences ensuring the initiation of transcription (e. g., translation initiation codon, promoters, such as naturally-associated or heterologous promoters and/or insulators; see above), internal ribosomal entry sites (IRES) (Owens, Proc. Natl. Acad. Sci. USA 98 (2001), 1471-1476) and optionally poly-A signals ensuring termination of transcription and stabilization of the transcript. Additional regulatory elements may include transcriptional as well as translational enhancers. Preferably, the polynucleotide(s) encoding the encoding the multispecific antibody of the invention is operatively linked to such expression control sequences allowing expression in prokaryotes or eukaryotic cells. The vector may further comprise nucleic acid sequences encoding secretion signals as further regulatory elements. Such sequences are well known to the person skilled in the art. Furthermore, depending on the expression system used, leader sequences capable of directing the expressed polypeptide to a cellular compartment may be added to the coding sequence of the polynucleotide of the invention. Such leader sequences are well known in the art.

Furthermore, it is preferred that the vector comprises a selectable marker. Examples of selectable markers include genes encoding resistance to neomycin, ampicillin, hygromycine, and kanamycin. Specifically designed vectors allow the shuttling of DNA between different hosts, such as bacteria-fungal cells or bacteria-animal cells (e. g. the Gateway system available at Invitrogen). An expression vector according to this invention is capable of directing the replication, and the expression, of the polynucleotide and encoded peptide or fusion protein of this invention. Apart from introduction via vectors such as phage vectors or viral vectors (e.g. adenoviral, retroviral), the nucleic acid molecules as described herein above may be designed for direct introduction or for introduction via liposomes into a cell. Additionally, baculoviral systems or systems based on vaccinia virus or Semliki Forest virus can be used as eukaryotic expression systems for the nucleic acid molecules of the invention.

The present invention relates in a fourth aspect to a host cell, preferably a non-human host cell comprising the vector of the invention.

The term “host cell” means any cell of any organism that is selected, modified, transformed, grown, or used or manipulated in any way, for the production of the protein or peptide or fusion protein of the invention by the cell. The host cell is therefore generally an ex vivo or in vitro cell and/or an isolated cell.

The host cell of the invention is typically produced by introducing the nucleic acid molecule(s) or vector(s) of the invention into the host cell which upon its/their presence mediates the expression of the nucleic acid molecule(s) of the invention encoding the multispecific antibody of invention. The host from which the host cell is derived or isolated may be any prokaryote or eukaryotic cell or organism, preferably with the exception of human embryonic stem cells that have been derived directly by destruction of a human embryo.

Suitable prokaryotes (bacteria) useful as hosts for the invention are, for example, those generally used for cloning and/or expression like E. coli(e.g., E coli strains BL21, HB101, DH5a, XL1 Blue, Y1090 and JM101), Salmonella typhimurium, Serratia marcescens, Burkholderia glumae, Pseudomonas putida, Pseudomonas fluorescens, Pseudomonas stutzeri, Streptomyces lividans, Lactococcus lactis, Mycobacterium smegmatis, Streptomyces coelicolor or Bacillus subtilis. Appropriate culture mediums and conditions for the above-described host cells are well known in the art.

A suitable eukaryotic host cell may be a vertebrate cell, an insect cell, a fungal/yeast cell, a nematode cell or a plant cell. The fungal/yeast cell may a Saccharomyces cerevisiae cell, Pichia pastoris cell or an Aspergillus cell. Preferred examples of a host cell to be genetically engineered with the nucleic acid molecule or the vector(s) of the invention is a cell of yeast, E. coli and/or a species of the genus Bacillus (e.g., B. subtilis). In one preferred embodiment the host cell is a yeast cell (e.g. S. cerevisiae).

In a different preferred embodiment the host cell is a mammalian host cell, such as a Chinese Hamster Ovary (CHO) cell, mouse myeloma lymphoblastoid, human embryonic kidney cell (HEK-293), human embryonic retinal cell (Crucell's Per.C6), or human amniocyte cell (Glycotope and CEVEC). The cells are frequently used in the art to produce recombinant proteins. CHO cells are the most commonly used mammalian host cells for industrial production of recombinant protein therapeutics for humans.

The present invention also relates to a transgenic animal, preferably a non-human transgenic animal comprising the vector of the invention.

Transgenic animals can be used for the production of antibodies as is reviewed, for example, in Bruggemann (2015), Arch Immunol Ther Exp (Warsz); 63(2): 101-108. The transgenic animal is preferably a mammal other than human. The antibodies may also be produced such that the antibodies can be obtained from the milk of transgenic mammals. The mammal is therefore preferably a goat, sheep or cow.

The present invention relates in a fifth aspect to a method for producing the multispecific antibody of the invention comprising (a) culturing the host cell of the invention under conditions where the host cell expresses the multispecific antibody of the invention, and (b) isolating the multispecific antibody of the invention as expressed in (a).

The term “culturing” specifies the process by which host cells are grown under controlled conditions. These conditions may vary dependent on the host cell used. The skilled person is well aware of methods for establishing optimized culturing conditions. Moreover, methods for establishing, maintaining and manipulating a cell culture have been extensively described in the state of the art.

Methods of isolation of the multispecific antibody of the invention are well-known in the art and comprise without limitation method steps such as ion exchange chromatography, gel filtration chromatography (size exclusion chromatography), affinity chromatography, high pressure liquid chromatography (HPLC), reversed phase HPLC, disc gel electrophoresis or immunoprecipitation, see, for example, Antibody Purification Handbook, GE Healthcare, 18-1037-46.

The term “the multispecific antibody of the invention as expressed in (a)” in accordance with the invention refers to the product of a process implying, that in the host cell a process can be induced by which information from nucleic acid molecule(s) encoding the multispecific antibody of the invention is/are used in the synthesis of the multispecific antibody of the invention. Several steps in this process may be modulated, including the transcription, RNA splicing, translation, and post-translational modification of the multispecific antibody of the invention by methods know in the art. Accordingly, such modulation may allow for control of the timing, location, and amount of multispecific antibody produced.

The present invention relates in a sixth aspect to a pharmaceutical composition comprising the multispecific antibody of the invention, the nucleic acid sequence of the invention, the vector of the invention or the host cell of the invention, and optionally comprising (a) an antibody specifically binding to an antigen being expressed on the surface of a tumor cell other than the antigen of (i), wherein the antibody of (a) preferably specifically binds to CD19 or CD38, and/or (b) an antibody specifically binding to an antigen being expressed on the surface of a cytotoxic lymphocyte other than the antigen of (ii), wherein the antibody of (b) preferably specifically binds to CD3 as expressed on the surface of T cells and NKT cells and/or CD16 or CD32 as expressed on the surface of NK cells, and/or (c) a cell product, preferably chimeric antigen receptor T cells or chimeric antigen receptor natural killer cells, wherein said cell product expresses the antigen of (ii), and wherein said cell product preferably comprises cytotoxic lymphocytes that are preferably genetically modified to express a synthetic immune receptor containing binding sites to an antigen other than that of (i).

In accordance with the present invention, the term “pharmaceutical composition” relates to a composition for administration to a patient, preferably a human patient. The pharmaceutical composition of the invention comprises the compounds recited above. It may, optionally, comprise further molecules capable of altering the characteristics of the compounds of the invention thereby, for example, stabilizing, modulating and/or activating their function. The composition may be in solid, liquid or gaseous form and may be, inter alia, in the form of (a) powder(s), (a) tablet(s), (a) solution(s) or (an) aerosol(s). The pharmaceutical composition of the present invention may, optionally and additionally, comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, organic solvents including DMSO etc. Compositions comprising such carriers can be formulated by well known conventional methods. These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The therapeutically effective amount for a given situation will readily be determined by routine experimentation and is within the skills and judgement of the ordinary clinician or physician. Generally, the regimen as a regular administration of the pharmaceutical composition should be in the range of 1 μg to 5 g units per day. However, a more preferred dosage might be in the range of 0.0001 mg to 100 mg/kg bodyweight, even more preferably 0.01 mg to 50 mg/kg bodyweight and most preferably 20 mg to 50 mg/kg bodyweight per day. The length of treatment needed to observe changes and the interval following treatment for responses to occur vary depending on the desired effect. The particular amounts may be determined by conventional tests, which are well known to the person skilled in the art.

The antibodies being optionally present in the pharmaceutical composition of the invention may either exert an immune activator function by binding to an antigen expressed by a tumor or an autoreactive immune cell (antibody (a)) and/or engaging an activating receptor expressed by an immune cell (antibody (b)) and are enhanced in their function through the immune modulator function of the multispecific antibody of the invention. In particular, the example herein below shows that the anti-CD38 antibody in combination with a multispecific antibody of the invention (CD20×NKG2D clone 3 or CD20×NKG2D clone 32) was significantly more effective in triggering cell-mediated killing of tumor cell than one of the two antibodies alone.

The term “cell product” preferably designates a cell therapeutic composition comprising native or genetically engineered cytotoxic leukocytes, preferably human cytotoxic leukocytes, such as CAR-T cells, CAR-NK cells or TILs. The cells are generally used for adoptive transfer into a patient. Since NKG2D is also expressed on ex vivo expanded NK cells and T cells (CAR-T, CAR-NK cells or TILs), the cytotoxic activity of adoptively transferred cells could be modulated by NKG2D-targeting molecules. By NKG2D stimulation the cytotoxic activity of the transferred cells might be enhanced.

The present invention relates in a seventh aspect to the multispecific antibody of the invention, the nucleic acid sequence of the invention, the vector of the invention, the host cell of the invention or the pharmaceutical composition of the invention for use in treating or preventing a tumor or an autoimmune disease.

It is to be understood that in connection with this aspect of the invention the multispecific antibody specifically binds to an antigen being expressed on the surface of a tumor cell if a tumor is to be treated or prevented. Similarly, the multispecific antibody specifically binds to an antigen being expressed on the surface of an autoreactive immune cell if an autoimmune disease is to be treated or prevented.

In the examples herein below it is shown that the multispecific antibody of the invention effectively and highly specifically mediates the killing of tumor cells. The multispecific antibody of the invention is therefore suitable to treat a tumor in a patient. The selective killing of tumor cells also renders it at least plausible that also autoimmune diseases can be treated by the multispecific antibody of the invention, since also autoimmune diseases are mediated by specific populations of cells and the removal of these cells will have a curative or preventive effect.

The tumor can be a benign or a malignant tumor. The tumor is preferably a malignant tumor and a malignant tumor is also referred to herein as cancer.

In accordance with a preferred embodiment of the seventh aspect of the invention in addition (a) an antibody specifically binding to an antigen being expressed on the surface of a tumor cell other than the antigen of (i) is used, wherein the antibody of (a) preferably specifically binds to CD19 or CD38, and/or (b) an antibody specifically binding to an antigen being expressed on the surface of a cytotoxic lymphocyte other than the antigen of (ii) is used, wherein the antibody of (b) preferably specifically binds to CD3 as expressed on the surface of T cells or CD16 or CD32 as expressed on the surface of NK cells, and/or (c) a cell product, being preferably a chimeric antigen receptor T cell or a chimeric antigen receptor natural killer cell, wherein said cell product expresses the antigen of (ii), and wherein said cell product preferably comprises cytotoxic lymphocytes that are preferably genetically modified to express a synthetic immune receptor containing binding sites to an antigen expressed by tumor cells other than that of (i).

As discussed in connection with the sixth aspect of the invention, the antibodies being optionally present in the pharmaceutical composition of the invention may either exert an immune activator function by binding to an antigen expressed by a tumor or an autoreactive immune cell (antibody (a)) and/or engaging an activating receptor expressed by an immune cell (antibody (b)) and are enhanced in their function through the immune modulator function of the multispecific antibody of the invention. Also the cell product has been defined in connection with the sixth aspect of the invention and the cell product it the same in accordance with the seventh aspect of the invention.

The present invention relates in a eighth aspect to an antibody, preferably a multispecific antibody comprising the six CDRs of SEQ ID NOs 1 to 5 and the CDR2 VL GNN, or SEQ ID NOs 6 to 10 and the CDR2 VL GKN, or SEQ ID NOs 11 to 15 and the CDR2 VL GKN, and preferably comprising the variable heavy and light chain regions of SEQ ID NOs 16 and 17 or SEQ ID NOs 18 and 19 or SEQ ID NOs 20 and 21.

This antibody is an anti-NKG2D antibody. As discussed herein above, 36 anti-NKG2D antibodies were processed into Fab-scFv fusion proteins, wherein the Fab fragment is directed against CD20 and the scFv fragment is directed against NKG2D. From these 36 Fab-scFv fusion proteins in particular the three clones with the anti-NKG2D antibodies 3, 32 and 35 as referred to in the examples were capable of inducing potent NK cell activation.

Since NKG2D in humans is expressed by NK cells, NK1.1+ T cells, γδ T cells and CD8+ αβ T cells it is believed that the anti-NKG2D antibodies 3, 32 and 35 are not only particularly advantageous to activate these cells in the format of multispecific antibody of the invention but that clones 3, 32 and 35 are generally outstandingly well performing anti-NKG2D antibodies.

Also described herein is an antibody comprising the variable heavy and light chain regions being at with increased preference at least 90%, at least 95%, at least 98% and at least 99% identical to SEQ ID NOs 16 and 17 or SEQ ID NOs 18 and 19 or SEQ ID NOs 20 and 21. In this connection it is preferred that such an antibody comprises the six CDRs of SEQ ID NOs 1 to 5 and the CDR2 VL GNN, or SEQ ID NOs 6 to 10 and the CDR2 VL GKN, or SEQ ID NOs 11 to 15 and the CDR2 VL GKN with no changes.

The present invention relates in a ninth aspect to the antibody of the eighth aspect for use in the treatment of a tumor, an autoimmune disease, an inflammatory disease or graft versus host disease.

Anti-NKG2D antibodies are known to be suitable for the treatment of diseases, such as a tumor, an autoimmune disease, an inflammatory disease or graft versus host disease. Various studies have demonstrated the antitumor function mediated by NKG2D on natural killer cells and on conventional and unconventional T cells. NKG2D controls tumor growth and infections (Shepppard et al., Front Immunol. 2018; 9: 1808). Anti-NKG2D antibodies were used in clinical trials for the treatment of Crohn's disease (CD) and ulcerative colitis (UC) (Vadstrup and Bendtsen, Int J Mol Sci. 2017 September; 18(9): 1997).

Regarding the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all appended claims.

The figures show.

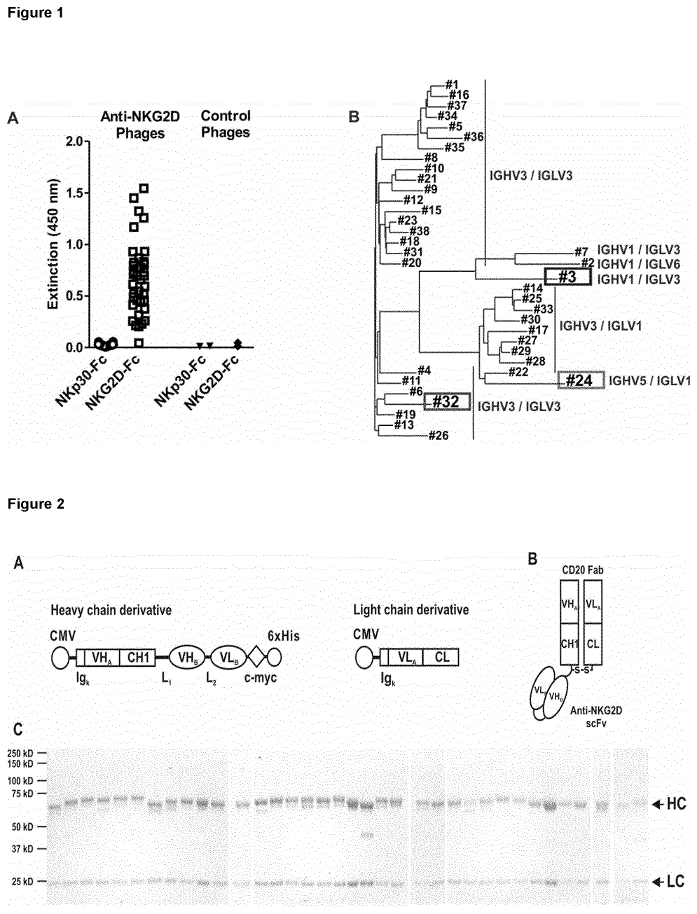

FIG. 1. Isolation and sequence analysis of NKG2D-binding scFvs. (A) ScFv phages, which were isolated from a naïve antibody library by panning against human NKG2D antigen. Specificity of binding was analyzed by phage ELISA using the NKG2D-Fc-fusion protein and the analogously constructed control protein NKp30-Fc. Irrelevant phages were used as an additional control. (B) The isolated NKG2D-specific scFvs were grouped via sequence analysis into 3 different groups according to different germline gene segment families as well as their VH/VL combinations. The further characterized clones #3 (blue) and #32 (red) and the later used control scFv #24 (green) are highlighted.

FIG. 2. Production and characterization of bispecific [CD20×NKG2D] antibodies. (A) Schematic illustration of the expression cassettes for the production of bispecific [CD20×NKG2D] antibodies in the bibody format. CMV, cytomegalovirus promotor; Ig_{κ}, human Ig kappa secretion leader; VH_{A}, VL_{A}, sequences coding for the variable regions of the immunoglobulin heavy and light chains of the anti-CD20 antibody rituximab, respectively; CH1, CL, sequences coding for the human immunoglobulin heavy chain constant region 1 and the human immunoglobulin kappa-light chain constant region, respectively; VH_{B}, VL_{B}, cDNA sequence coding for the variable heavy and light chain regions of the NKG2D-specific scFv; L₁, L₂, sequence coding for a linker peptide; c-myc, 6×His, sequence coding for the c-myc epitope and a hexahistidine tag, respectively. (B) Block structure of the bispecific bibody format. The NKG2D-specific scFvs were genetically fused to a CD20 directed Fab. S-S, disulfide bridge. (C) Purity and integrity of purified bispecific antibodies, consisting of a light chain (LC, ca. 25 kDa) and a heavy chain derivate (HC, 70-75 kDa), were analyzed by Coomassie stained SDS-PAGE under reducing conditions. One representative experiment out of three is shown.

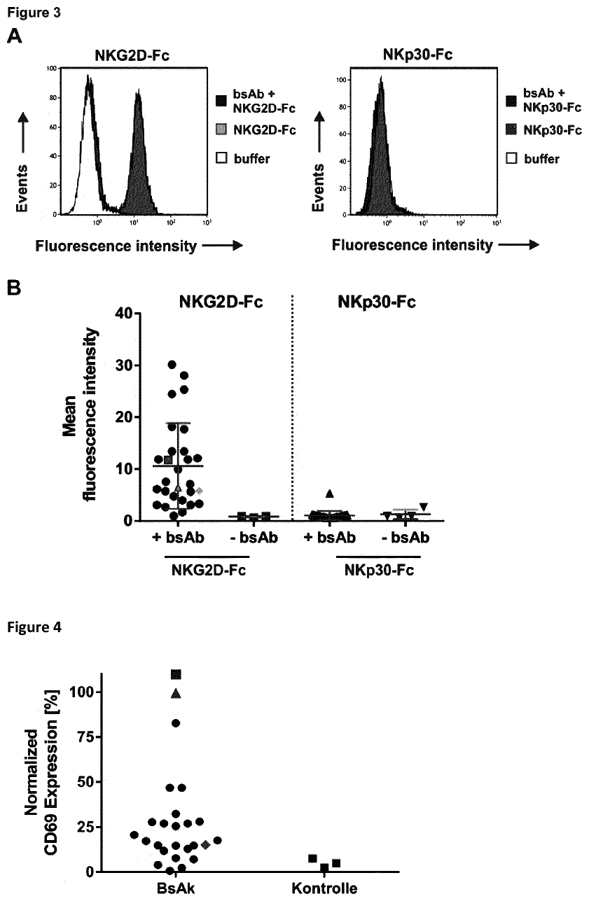

FIG. 3. Simultaneous antigen binding of bispecific [CD20×NKG2D] antibodies (A) CD20 positive Raji lymphoma cells were initially incubated with the different bispecific [CD20×NKG2D] antibodies, which then reacted in a following incubation step either with a fusion protein containing the extracellular domain of NKG2D fused to the human IgG1 Fc-portion (NKG2D-Fc) or with the control protein NKp30-Fc. Binding was visualized by a fluorescence-coupled antibody against human Fc via flow cytometry. Detection is only possible by simultaneous binding of cellular CD20 as well as soluble NKG2D. As a control the cells were incubated with the Fc-fusion proteins in absence of [CD20×NKG2D] bsAbs (NKG2D-Fc; NKp30-Fc) or with the FITC-coupled detection antibody alone (buffer). The results are shown exemplary for the bsAb [CD20×NKG2D #3], which interacts only with NKG2D-Fc but not with NKp30-Fc. (B) Abilities of various individual [CD20×NKG2D] bsAbs to bind CD20 and NKG2D simultaneously. Each data point represents an individual clone. The further characterized clones #3 (square) and #32 (triangle) and the later used control scFv #24 (diamond) are highlighted. Data are representative of three independent experiments.

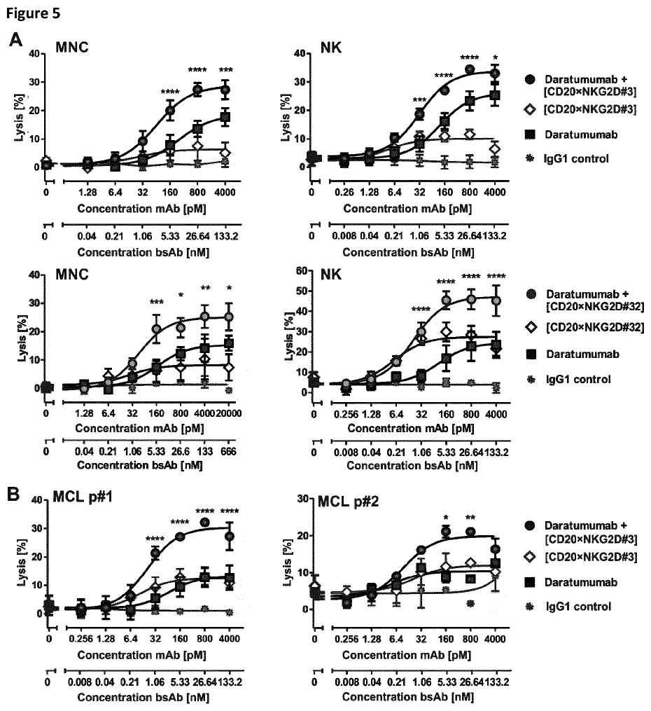

FIG. 4. NK cell activation with bispecific [CD20×NKG2D] antibodies. NK cells were incubated with the [CD20×NKG2D] bsAbs (10 μg/ml) in the presence of GRANTA-519 mantle cell lymphoma cells. After 4 h the induced expression of the early activation marker CD69 was analyzed on CD56+/CD3− NK cells via flow cytometry. NK cells and lymphoma cells were incubated in absence of the bsAbs as a control. The further characterized clones #3 (square) and #32 (triangle) and the later used control scFv #24 (diamond) are highlighted. Data points were normalized to CD69 expression induced by clone #3 and indicate mean values from 3 independent experiments.

FIG. 5. Cytotoxicity of the bispecific antibodies [CD20×NKG2D #3] and [CD20×NKG2D #32] and synergy with CD38 antibody daratumumab (A) CD20⁺/CD38⁺ GRANTA-519 MCL cells were incubated with daratumumab, the bispecific [CD20×NKG2D] antibodies or their combinations, respectively, in presence of mononuclear cells (MNC; E:T ratio: 40:1) or NK cells (E:T ratio: 10:1) as effector population. After 4 h, lysis of target cells was analyzed. The data points represent mean values of three independent experiments ±SEM. (*, statistically significant differences to treatment with daratumumab only; P≤0.05). (B) [CD20×NKG2D #3] enhances ADCC triggered by daratumumab against tumor cells derived from two different MCL patients (p). NK cells were used as effector population. Data points represent mean values from two independent experiments±SEM. (*, statistically significant differences to treatment with daratumumab only; ≤0.05).

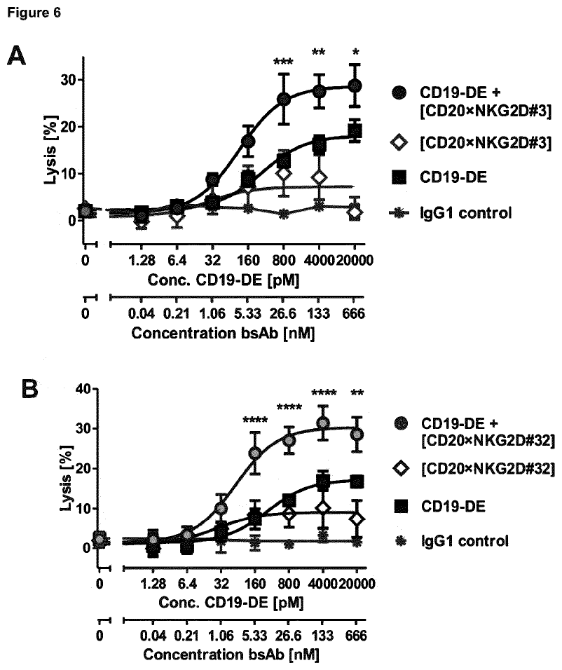

FIG. 6. Cytotoxicity of the bispecific antibodies [CD20×NKG2D #3] and [CD20×NKG2D #32] and synergy with an Fc-engineered CD19 antibody (CD19-DE), respectively. The cytotoxic function of the bispecific antibodies [CD20×NKG2D #3] (A) and [CD20×NKG2D #32] (B) alone, or in combination with the Fc-engineered CD19-DE antibody, was analyzed in a 4 h ⁵¹Cr release assay. GRANTA-519 MCL cells (CD19⁺, CD20⁺) were used as target cells and MNC isolated from healthy donors were applied as effector population (E:T ratio: 40:1). A non-binding monoclonal IgG1 Ab was used as a control. The data points represent the mean value of three independent experiments ±SEM. (*, statistically significant differences compared to treatment with CD19-DE only; ≤0.05).

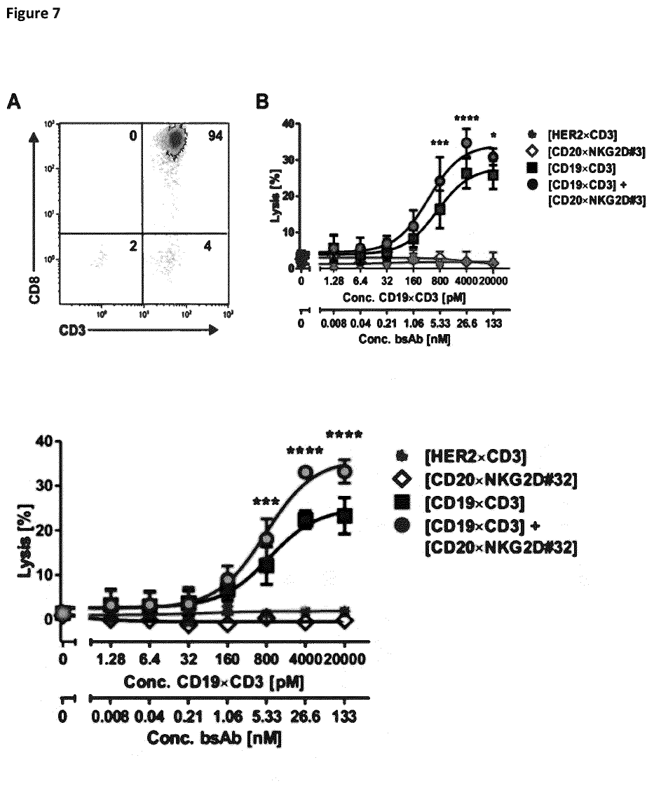

FIG. 7. Cytotoxic activity of bispecific [CD20×NKG2D] antibodies with CD8-positive αβ T cells as effector population. (A) CD8⁺ αβ T cells were isolated via MACS. The purity was determined by flow cytometry using CD3, CD8, CD16 and CD56 antibodies labelled with appropriate fluorescent dyes. Shown is the histogram for the CD3⁺/CD8⁺ cells (upper right rectangle), with a relative amount of 94% in this representative experiment. (B) The purified T cells were stimulated with interleukin-2 (300 U/ml) for 3 days and were tested as effector cells (E:T ratio: 20:1) for the bispecific antibodies [CD20×NKG2D #3] and [CD20×NKG2D #32] as well as their combinations with a [CD19×CD3] bsscFv in a 4 h ⁵¹Cr release assay. GRANTA-519 MCL cells were used as target cells. The data points represent the mean value of three independent experiments ±SEM. (*, statistically significant differences against the treatment with [CD19×CD3] only; ≤0.05). A bispecific scFv [HER2×CD3] was used as a control.

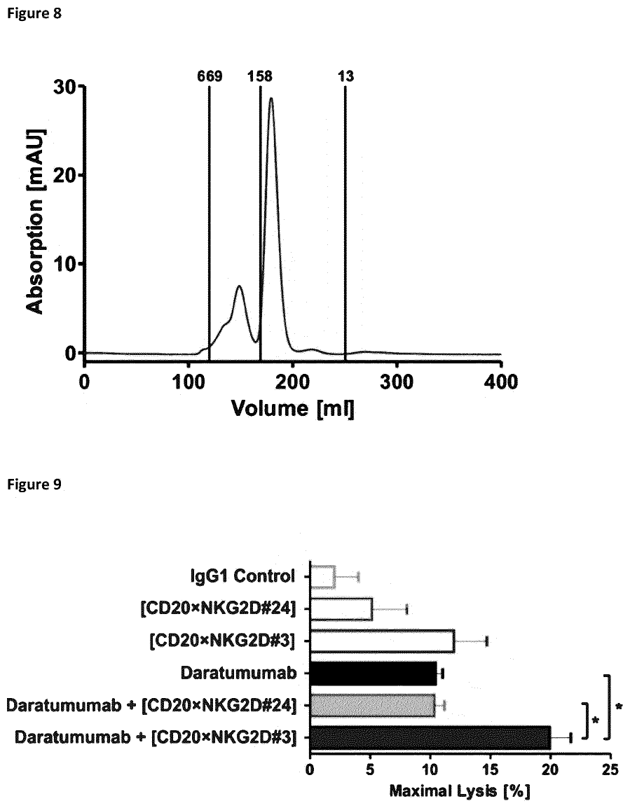

FIG. 8. Size exclusion chromatography (SEC) of bispecific antibody [CD20×NKG2D #32]. Bispecific antibody [CD20×NKG2D #32] was analyzed by SEC and compared to selected mass standards (669 kDa, 158 kDa, 13 kDa). One representative experiment is shown.

FIG. 9. Cytotoxicity of the bispecific antibodies [CD20×NKG2D #3] and [CD20×NKG2D #24] and synergy with daratumumab, respectively. The cytotoxic function of the bispecific antibodies [CD20×NKG2D #3] and [CD20×NKG2D #24] alone or in combination with daratumumab, was analyzed in a 4 h ⁵¹Cr release assay. Tumor cells derived from MCL patients (CD38⁺, CD20⁺) were used as target cells and NK cells isolated from healthy donors as effector population (E:T ratio: 10:1). A non-binding monoclonal IgG1 Ab was used as a control. The data points represent the mean value of three independent experiments ±SEM. (*, statistically significant differences are indicated; p≤0.05).

FIG. 10. Cytotoxicity of combinations of bispecific [4D5×NKG2D #32] with Cetuximab. The cytotoxic function of the bispecific antibody [4D5×NKG2D #32] (B) alone, or in combination with Cetuximab, was analyzed in 4 h 51Cr release assays. Cetuximab and 4D5×NKG2D #32 were applied in a molar ratio of 1:1000. SKBR3 cells (Her2+, EGFR+, CD20−) were used as target cells and MNC isolated from healthy donors were applied as effector population (E:T ratio: 40:1). A non-binding monoclonal IgG1 Ab was used as a control. The data points represent the mean value of triplicate wells ±SEM.

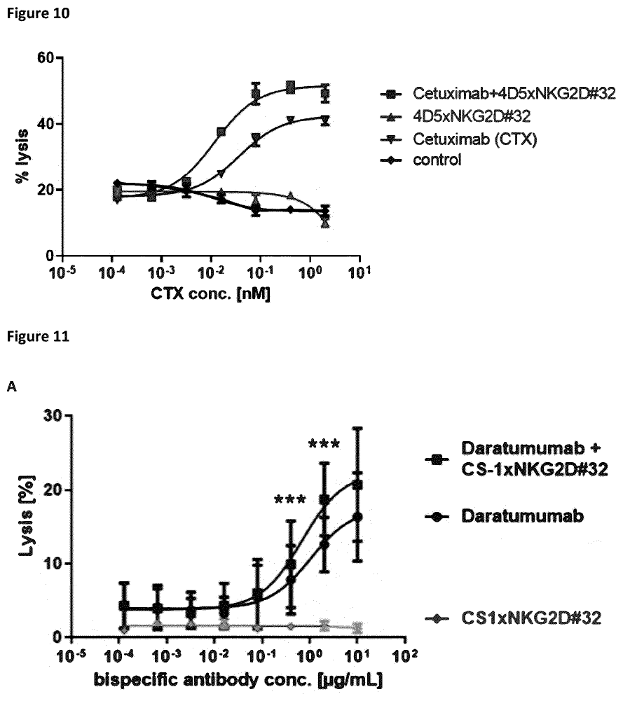

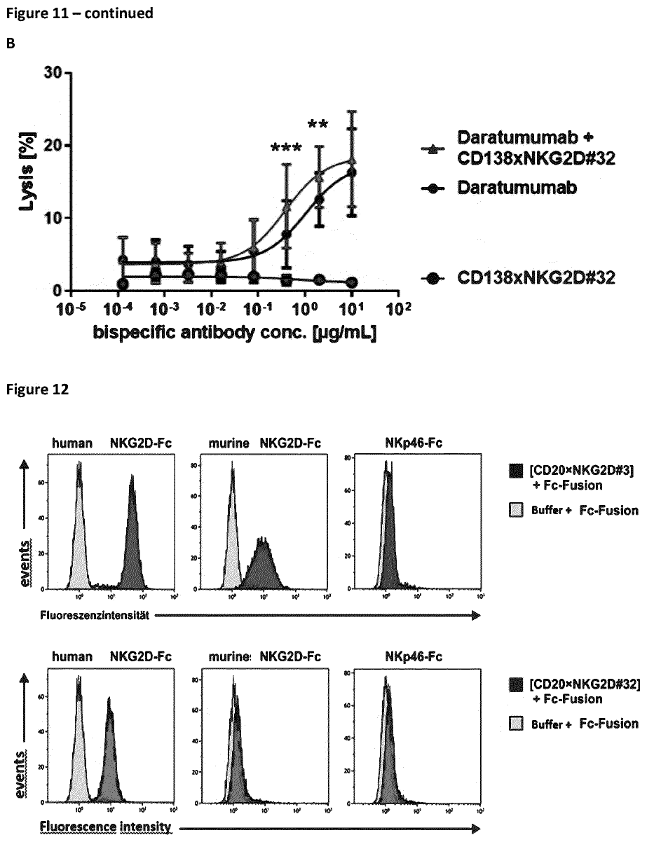

FIG. 11. Cytotoxicity of combinations of bispecific [CS1×NKG2D #32], [CD138×NKG2D #32] antibodies with Daratumumab. The cytotoxic function of the bispecific antibody [CS1×NKG2D #32] (A) or [CD138×NKG2D #32] (B) in combination with Daratumumab, was analyzed in 4 h 51Cr release assays. Daratumumab and the bispecific antibodies were applied in a molar ratio of 1:16.6. L363 cells (CS1+, CD38+, CD138+) were used as target cells and MNC isolated from healthy donors were applied as effector population (E:T ratio: 20:1). The data points represent the mean value of 4 independent experiments ±SEM.

FIG. 12. Cross-reactivity of the bispecific [CD20×NKG2D] antibodies with murine NKG2D. CD20-positive Ramos Burkitt lymphoma cells were incubated with the bispecific [CD20×NKG2D] antibodies and then with a fusion protein of the extracellular domain of murine NKG2D and the human IgG1 Fc domain (murine NKG2D-Fc) or for comparison with the human NKG2D-Fc fusion protein (human NKG2D-Fc) or the control protein NKp46-Fc ([CD20×NKG2D]+Fc fusions). The binding of the fusion proteins was then detected with a FITC-coupled antibody against human Fc domain analyzed in flow cytometer. As a result, the bispecific antibody [CD20×NKG2D #3] reacted with both human NKG2D (left, top) and murine NKG2D (middle, top), but not with the control protein (right). The antibody [CD20×NKG2D #32], on the other hand, only showed a reaction with the human NKG2D fusion protein (left, bottom).

The Examples illustrate the invention.

### EXAMPLE 1—MATERIAL AND METHODS

### Phage Display

Phage display experiments were performed as described previously [29]. Naïve antibody gene libraries HAL7 and HAL7b were used for bio-panning against recombinant human NKG2D-Fc fusion protein. Analogous constructed NKp30-Fc was employed as a control.

### Sequencing, Sequence Analysis

Sanger sequencing was used to identify different NKG2D-specific scFvs and to verify DNA sequences. Sequence analyzes were done using VBASE2 and Vector NTI Advance 11.5.4 software.

### Cell Culture.

Raji cells (DSMZ) were maintained in RPMI 1640 Glutamax-I medium (Invitrogen) supplemented with 10% fetal calf serum (FCS; Invitrogen), 100 U/mL penicillin and 100 mg/mL streptomycin (Invitrogen). GRANTA-519 (DSMZ) and Lenti-X 293T cells (Takara Bio Europe/Clontech) were cultured in Dulbecco's modified Eagle medium-Glutamax-I medium (Invitrogen) supplemented with 10% FCS, 100 U/mL penicillin and 100 μg/mL streptomycin. Chinese hamster ovary (CHO)-S, suspension-adapted CHO cells (Life Technologies) were kept in CD CHO-Medium (Gibco/Invitrogen) containing 1% GlutaMax (200 mM L-Ala-L-Gln, Gibco/Invitrogen) and 1% HT Supplement for maintenance and in CD OptiCHO (Gibco) supplemented with 1% Pluronic-F68, 1% GlutaMax and 1% HAT-Supplement (CHO production medium) for antibody production.

Cloning, Expression and Purification of bsAbs and Antibody-Derivatives.

For construction of the heavy chain derivatives of bispecific bibodies, DNA sequences for the different anti-NKG2D scFv were ligated as NcoI/NotI cassettes into expression vector pΔIRES-RTX-VH-CH1 (M. Peipp, unpublished), a derivative of vector pIRES-ZSK Green in which both the internal ribosomal entry site and the GFP coding sequence had been replaced by sequences coding for the rituximab VH leader, rituximab VH chain, the IgG1 CH1 domain and the antibody's upper hinge region. For production of small quantities for [CD20×NKG2D] bsAb screening, Lenti-X 293T cells were transiently co-transfected with expression vectors encoding either the bibodies' heavy chain derivative or the rituximab light chain [30] by the calcium phosphate method [9]. Selected clones were also expressed transiently in CHO-S cells by flow electroporation using MaxCyte STX electroporation system (MaxCyte) as described previously [31]. Afterwards, cells were cultured in CHO production medium (REF) at 32° C., 5% CO₂ and 143 rpm until cell viability decreased below 50%. Feed stock solution, which contains 70% CHO CD Efficient Feed A Stock Solution (Invitrogen), 14% Yeastolate TC UF (Becton Dickinson), 3.5% GlutaMax (200 mM) and 12.5% Glucose (450 g/L, Sigma), was supplemented daily. Cell culture supernatants were collected and proteins were purified by affinity chromatography with CaptureSelect IgG-CH1 affinity matrix (Thermo Fisher Scientific) following manufacturer's instructions. BsscFvs [CD19×CD3] and [HER2×CD3], which both based on the CD3 scFv moiety from blinatumomab (WO2005/040220), were expressed and purified as described previously [32]. Fusion proteins NKG2D-Fc and NKp30-Fc were produced as previously published [33]. After extensive dialysis against phosphate-buffered saline (PBS, Invitrogen) the molecules were stored at 4° C. until usage.

### SDS-PAGE.

Separation and detection of recombinant bsAbs were performed by sodium dodecyl sulfate polyacrylamide gel electrophoresis (SDS-PAGE) under reducing or non-reducing conditions, according to standard procedures. Quantity and quality of the proteins were analyzed by Coomassie staining (Coomassie brilliant blue G250 solution, Carl Roth GmbH). The concentration of purified antibodies was estimated against a standard curve of rituximab (Roche).

### Flow Cytometry.

Flow cytometry experiments were performed on Navios flow cytometer (Beckman Coulter). Three hundred thousand cells were washed in PBS supplemented with 1% bovine serum albumin (Sigma-Aldrich) and 0.1% sodium-azide (PBA). Simultaneous binding was demonstrated by preincubating Raji cells with the [CD20×NKG2D] bsAbs (50 μg/mL), followed by a second incubation step with either NKG2D-Fc (100 μg/mL) or the control protein NKp30-Fc on ice for 60 minutes. Finally, the surface-bound complex was detected by staining with polyclonal FITC-coupled anti-human IgG-Fc F(ab′)₂ fragments (Beckman Coulter). Isolated NK or T cells were characterized by flow cytometry using FITC or Pacific Blue-conjugated CD3 antibodies, APC-coupled CD56 antibodies, PE-conjugated CD16 antibodies (Beckman Coulter) and corresponding isotype controls according to the manufacturer's recommendations. CD19, CD20 and CD38 expression on target cells was analyzed analogously using PE or FITC-conjugated antibodies (Beckman Coulter).

### Preparation of MNC and Isolation of NK and T Cells.

All experiments were authorized by the Ethics Committee of the Christian-Albrechts-University of Kiel (Kiel, Germany). Blood from healthy donors was drawn after receiving written informed consent. Preparation of MNC from peripheral blood of patients and healthy volunteers or from leukocyte reduction system chambers was performed via Ficoll-Paque PLUS density gradient (GE Healthcare). After centrifugation, MNC were collected at the Serum/Ficoll interface and remaining erythrocytes were removed by hypotonic lysis. NK cells and CD8-positive αβ T cells were isolated from MNC by MACS technology via negative selection using NK cell isolation kit and CD8⁺ T cell isolation kit (Miltenyi), respectively, following the manufacturer's protocols. Purified MNC were directly employed in functional assays. Enriched NK cells were cultured overnight at a density of 2×10⁶ cells/mL in RPMI 1640 Glutamax-I medium supplemented with 10% FCS, 100 U/mL penicillin and 100 mg/mL streptomycin. CD8⁺ T cells were stimulated with IL-2 (300 U/mL) for 48 h before using them in functional assays. Cells were kept at a density of 1×10⁶ cells/mL in RPMI 1640 Glutamax-I medium supplemented with 10% FCS, 100 U/mL penicillin and 100 mg/mL streptomycin.

### Analysis of NK Cell Activation.

One hundred thousand NK cells were incubated together with equal numbers of GRANTA-519 cells in microtiter plates in a volume of 200 μL. The [CD20×NKG2D] bsAbs, rituximab (Roche), trastuzumab (Roche) or PBS were added. After 4 h cells were stained with antibodies against CD69 (PE-conjugated, Beckman Coulter), CD56 (APC, Beckman Coulter), CD19 (FITC, Beckman Coulter) and CD3 (Pacific Blue, Beckman Coulter) and analyzed by flow cytometry. CD56-positive, CD3- and CD19-negative NK cells were gated and the expression levels of CD69 were determined.

### Analysis of NK Cell and T Cell Cytotoxicity.

Cytotoxicity was analyzed in standard 4 h ⁵¹Cr release experiments, which were performed in 96-well microtiter plates in a total volume of 200 μL as described previously [9]. Human NK cells, CD8⁺ T cells or MNC were used as effector populations at the indicated effector-to-target cell (E:T) ratios.

### Statistical Analysis and Data Processing.

P-values were determined using repeated measures ANOVA and the Bonferroni post-test. The null hypothesis was rejected for p<0.05. Statistical and graphical analyses were performed with GraphPad Prism 5.0 software. Synergy was analyzed by interpolating required antibody doses at distinct effect levels using GraphPad Prism 5.0 software and calculating combination index (CI) values using the formula CIₓ=D_{A}/D_{xA}+D_{B}/D_{xB} (D_{xA} and D_{xB}, dose of drugs A and B alone producing x % effect; D_{A} and D_{B}, doses of drugs A and B in combination producing equal effects) [34]. Synergistic effects were classified into strong synergy (CI=0.1-0.3), synergy (CI=0.3-0.7), moderate synergy (CI 0.7-0.85), slight synergy (CI=0.85-0.95) and no antagonism (CI>1).

### EXAMPLE 2—ISOLATION OF HUMAN NKG2D ANTIBODIES BY PHAGE DISPLAY

To generate novel human NKG2D-specific antibodies a naïve human phage display scFv library [29] was screened by bio-panning against a recombinant fusion protein consisting of the extracellular domain of human NKG2D and the human IgG1-Fc domain. By this means 38 different phages were isolated which in enzyme-linked immunosorbent assays (ELISA) bound human NKG2D-Fc but not an analogously constructed control molecule containing the extracellular domain of NKp30 (FIG. 1 A). A detailed sequence analysis by aligning the variable regions of the different NKG2D-reactive scFvs revealed that the individual clones could be assigned to different families of germline gene segments, had various V_{H}/V_{L} combinations and could be divided into three groups. The largest group had combinations of IGHV3 and IGLV3 framework families (25 clones), followed by IGHV3/IGLV1 (9 clones). Furthermore, rare combinations of IGHV1/IGLV3 (2 clones), IGHV1/IGLV6 and IGHV5/IGLV1 (1 clone each) were identified. All analyzed V-regions belonged to the major human V_{H}- (V_{H}1-V_{H}6) and V_{L}-families (V_{κ}1-V_{κ}4; V_{λ}1-V_{λ}3) [35], except of IGLV6. As expected, IGHV3 occurred in the majority of all clones, since it is reported to be the domain with the highest thermodynamic stability and yield of soluble protein [36].

### EXAMPLE 3—PRODUCTION OF BISPECIFIC ANTIBODIES

The 38 isolated clones were processed into bispecific [CD20×NKG2D] antibodies. To ensure an efficient screening process we used the heterodimeric bibody format, which contained a fragment antigen binding (Fab) derived from the monoclonal CD20 antibody rituximab, genetically fused to the different anti-NKG2D scFvs via a flexible glycine-serine-linker (FIGS. 2A and B). The resulting bsAbs were transiently expressed and purified from cell culture supernatants via CH1-specific affinity chromatography. Thirty-six of the 38 individual anti-NKG2D scFvs were successfully produced in the bispecific format. For two clones protein expression was not feasible for yet unknown reasons. Integrity and purity of the proteins were analyzed in a Coomassie-stained SDS-PAGE (FIG. 2C). For selected experiments multimers were removed by size exclusion chromatography (FIG. 8).

### EXAMPLE 4—ANTIGEN BINDING

Next, the binding abilities of the different [CD20×NKG2D] antibodies were analyzed by flow cytometry. In particular, the capacity of simultaneous binding of the two antigens was analyzed, which is essential to crosslink target and effector cells. Therefore, CD20-positive lymphoma cells were first incubated with the bispecific [CD20×NKG2D] antibodies, and then reacted either with soluble human NKG2D-Fc or the control protein NKp30-Fc. Cell-bound Fc-fusion proteins were subsequently detected with an antibody against the human Fc domain. Detection of the whole complex was only possible when the bsAbs had bound cellular CD20 as well as soluble NKG2D-Fc simultaneously. As indicated by shifts in mean fluorescence intensity (MFI), the different NKG2D-specific bsAbs reacted with both CD20 and NKG2D, except of one clone (FIGS. 3A and 3B). Interestingly, the clones showed various MFI values, which likely may be due to different binding affinities to NKG2D. In contrast, after incubation with the control molecule NKp30-Fc no binding was detectable, confirming the specificity of the bsAbs for the extracellular domain of human NKG2D.

### EXAMPLE 5—NK CELL ACTIVATION

An important ability of effector cell-directed bsAbs is their capacity to activate the targeted immune effector cell population. Hence, the 36 different [CD20×NKG2D] bispecific antibodies were analyzed for their ability to activate human NK cells. Therefore, NK cells and lymphoma cells were incubated in the presence of the bispecific NKG2D-antibodies and induced expression of the early activation marker CD69 on CD56-positive NK cells was measured. Interestingly, the majority of bsAbs were not or only moderately effective (FIG. 4). However, three different NKG2D-scFv clones (#3, #32, #35) were identified which induced potent NK cell activation. In the following experiments, we focused on bsAbs containing anti-NKG2D scFv clones #3 (blue) and #32 (red), which showed the highest activation efficiency. Both clones had IGVL3 framework but differed in the VH domain framework (FIG. 1B). Clone #24 (green) was chosen as representative control for bsAbs with low activation profile.

### EXAMPLE 6—CYTOTOXIC CAPACITY AND SYNERGISTIC ACTIVITY IN COMBINATION WITH NATIVE AND FC-ENGINEERED ANTIBODIES

In previous studies we have shown that bispecific immunoligands engaging NKG2D trigger NK cell cytotoxicity and enhance NK cell-mediated ADCC by therapeutic antibodies [19, 20]. To investigate, whether the novel bispecific [CD20×NKG2D] antibodies exerted these functions, bsAbs were either analyzed as single agents or were combined with the CD38 antibody daratumumab, and cytotoxicity was analyzed with both mononuclear cells (MNC) and purified NK cells. CD38/CD20 double-positive mantle cell lymphoma (MCL) GRANTA-519 cells or isolated lymphoma cells from two MCL patients were used as target cells. Both bibodies [CD20×NKG2D #3] and [CD20×NKG2D #32] induced considerable lysis of GRANTA-519 MCL cells with purified NK cells as effector cell population as single agents. Furthermore, [CD20×NKG2D #3] was also able to mediate lysis of patient lymphoma target cells (FIG. 5B). However, when MNC were applied, no significant influence was observed with both bibodies on GRANTA-519 target cells (FIG. 5A). Importantly, the combination of the CD38 antibody and CD20-directed bsAbs [CD20×NKG2D #3] or [CD20×NKG2D #32] was significantly more effective in triggering effector cell-mediated killing of tumor cells than the single agents. This was the case both when the cell line GRANTA-519 (FIG. 5A) and isolated tumor cells from MCL-patients were analyzed (FIG. 5B). Interestingly, bsAbs containing a NKG2D-specific scFv such as clone #24 with low activation capacity in terms of CD69 induction were not able to increase tumor cell lysis in combination with monoclonal antibodies (FIG. 8).

Fc-engineering of mAbs by increasing their affinity to FcγRIIIa represents a powerful method to augment their cytotoxic potential [6]. In a previous study we have shown that increasing the affinity to FcγRIIIa beyond a certain threshold does not translate into a further gain in ADCC activity [37], revealing that ADCC by Fc engineered antibodies is difficult to enhance. To investigate whether this upper limit in NK cell-mediated ADCC is due to a certain maximum limit of FcγRIIIa activation or a more general limit in the cytotoxic capacity of NK cells, the cytolytic capacity of combinations of NKG2D-bispecific antibodies and an Fc-engineered antibody was analyzed. To address this question an Fc-engineered CD19 antibody (CD19-DE), which was modified for enhanced binding to activating FcγR [38], was tested in combination with the novel bispecific [CD20×NKG2D] antibodies in ADCC reactions. The co-stimulation of NKG2D by bsAbs even enhanced CD19-DE mediated ADCC against GRANTA-519 MCL target cells (FIG. 6), which express significant amounts of both CD19 and CD20 (data not shown). Thus, in comparison to CD19-DE as single agent, the maximum lysis was increased from 16.3±0.9% to 24.5±1.2% by clone #3 and from 17.2±1.7% to 28.2±2.2% by clone #32, respectively. Additionally, CD19-DE mediated ADCC was enhanced synergistically through NKG2D-engagement by both bsAbs. This was the case throughout all combination experiments using mAbs and the NKG2D-specific bsAbs (Tab. 1).

TABLE 1
CI values for the combinations of daratumumab or CD19-DE with the [CD20 × NKG2D]
bsAbs

| | | | CI values at lysis of | |
|---|---|---|---|---|
| Combination | Targets | Effectors | 5% | 10% |
| daratumumab + [CD20 × NKG2D#3] | GRANTA-519 | MNC | 0.31 | n.a. |
| daratumumab + [CD20 × NKG2D#3] | GRANTA-519 | NK cells | 0.68 | 0.17 |
| daratumumab + [CD20 × NKG2D#32] | GRANTA-519 | MNC | 0.42 | n.a. |
| daratumumab + [CD20 × NKG2D#32] | GRANTA-519 | NK cells | | |
| daratumumab + [CD20 × NKG2D#3] | MCL p#1 | NK cells | 0.59 | 0.24 |
| daratumumab + [CD20 × NKG2D#3] | MCL p#2 | NK cells | 2.47 | 0.22 |
| CD19-DE + [CD20 × NKG2D#3] | GRANTA-519 | MNC | 0.55 | n.a. |
| CD19-DE + [CD20 × NKG2D#32] | GRANTA-519 | MNC | 0.19 | n.a. |

Combination index (CI) was calculated from dose response curves using the indicated target and effector cells for two different effect levels using GraphPad Prism 5.0 software. Of note, when the indicated effect level was not reached by treatment with single agents at saturating concentrations CI values were not calculated (n.a., not applicable (Strong synergy CI = 0.1-0.3; synergy CI = 0.3-0.7; moderate synergy CI 0.7-0.85; slight synergy 0.85-0.95; antagonism CI > 1)

### EXAMPLE 7—CO-STIMULATION OF BISPECIFIC T CELL ENGAGERS

In contrast to its role as a primary activating receptor on NK cells, NKG2D is also expressed on CD8⁺ αβ− and γδ T cells, but has got a more complex function here. We demonstrated in previous studies, that bispecific NKG2D directed immunoligands were able to induce lysis of lymphoma cells by γδ T cell lines [39], but had low activity levels with peripheral blood αβ T cells [19]. To investigate the potential T cell stimulatory function of the novel bispecific antibodies, T cell-mediated tumor cell killing triggered by bsAbs [CD20×NKG2D #3] and [CD20×NKG2D #32] alone or in combination with a [CD19×CD3] bsscFv in a BiTE-like format was analyzed. Interestingly, both bsAbs were able to significantly increase T cell mediated lysis of GRANTA-519 MCL target cells in combination with the bsscFv [CD19×CD3], even though they had no measurable single agent activity (FIG. 7B).

In conclusion, two anti-NKG2D scFv clones were identified, that both when converted into bispecific antibodies were able to trigger NK cell cytotoxicity as single agent albeit at moderate levels, to enhance ADCC by native and Fc-engineered monoclonal antibodies synergistically, and to enhance CD3-directed bispecific antibodies in inducing tumor cell killing by CD8-positive T cells.

### EXAMPLE 8—COSTIMULATION OF TRASTUZUMAB IN BREAST CANCER

To evaluate whether the concept of combining NKG2D-directed bispecific antibodies and approved therapeutic antibodies is more generally applicable, [4D5×NKG2D #32], a bispecific antibody targeting the Her2 antigen on solid tumors was combined with the clinically approved antibody cetuximab. Mononuclear cells were used as effector cells and SKBR3 breast cancer cells as target cells. Similar to the results in the lymphoma model, also in this setting the bispecific antibody significantly enhanced the ADCC activity of Cetuximab (FIG. 10). Therefore, the proposed concept of using NKG2D-directed bispecific as enhancers of already approved immunotherapeutic agents is broadly applicable to various antigens being expressed on the surface of a tumor cell or an autoreactive immune cell.

4D5 (Trastuzumab) VH nucleotide sequence:
(SEQ ID NO: 30)
gaggttcagctggtggagtctggcggtggcctggtgcagccagggggctcactccgtttgtcctgtgcagcttctggcttcaacattaaagac
acctatatacactgggtgcgtcaggccccgggtaagggcctggaatgggttgcaaggatttatcctacgaatggttatactagatatgccgat
agcgtcaagggccgtttcactataagcgcagacacatccaaaaacacagcctacctgcagatgaacagcctgcgtgctgaggacactgccgtc
tattattgttctagatggggaggggacggcttctatgctatggactattggggtcaaggaaccctggtcaccgtctcctcg

### 4D5 (Trastuzumab) VH Amino Acid Sequence:

evqlvesggglvqpggslrlscaasgfnikdtyihwvrqapgkglewvariyptngytryadsvkgrftisadtskntaylqmnslraedtavyyc srwggdgfyamdywgqgtlvtvss (SEQ ID NO: 31)

CDR1 VH: GFNI (SEQ ID NO: 32)

CDR2 VH: IYPTNGYT (SEQ ID NO: 33)

CDR3 VH: SRWGGDGFYAMDY (SEQ ID NO: 34)

4D5 (Trastuzumab) VL nucleotide sequence:
(SEQ ID NO: 35)
gatatccagatgacccagtccccgagctccctgtccgcctctgtgggcgatagggtcaccatcacctgccgtgccagtcaggatgtgaatac
tgctgtagcctggtatcaacagaaaccaggaaaagctccgaaactactgatttactcggcatccttcctctattctggagtcccttctcgct
tctctggatccagatctgggacggatttcactctgaccatcagcagtctgcagccggaagacttcgcaacttattactgtcagcaacattat
actactcctcccacgttcggacagggtaccaaggtggagatcaaa

### 4D5 (Trastuzumab) VL Amino Acid Sequence:

diqmtqspssisasvgdrvtitcrasqdvntavawyqqkpgkapklliysasflysgvpsrfsgsrsgtdftltisslqpedfatyycqqhyttpptfg qgtkveik (SEQ ID NO: 36)

CDR1 VL: QDVNTA (SEQ ID NO: 37)

CDR2 VL: SAS

CDR3 VL: QQHYTTPPT (SEQ ID NO: 38)

### EXAMPLE 9—COSTIMULATION OF DARATUMUMAB IN MULTIPLE MYELOMA

To further evaluate whether the concept of combining NKG2D-directed bispecific antibodies and approved therapeutic antibodies is generalizable, [CS1×NKG2D #32] or [CD138×NKG2D #32], bispecific antibodies targeting CS1 (CD319) or CD138 in Multiple Myeloma were combined with the clinically approved antibody Daratumumab. Mononuclear cells were used as effector cells and SKBR3 breast cancer cells as target cells. Similar to the results in the lymphoma model and breast cancer model also in this setting the bispecific antibodies significantly enhanced the ADCC activity of Daratumumab (FIG. 11).

CD138 VH nucleotide sequence:
(SEQ ID NO: 39)
caggtgcagctgcagcagtctggatccgagctgatgatgcctggggcctcagtgaagatatcctgcaaggctactggctacacattcagtaa
ctactggatagagtgggtaaagcagaggcctggacatggccttgagtggattggagagattttacctggaacaggtaggactatatacaatg
agaagttcaagggcaaggccacattcactgcagatatttcctccaacacagtccagatgcaactcagcagcctgacatctgaggactctgcc
gtctattactgtgcaagaagggactattacggcaacttctactatgctatggactactggggccaagggaccagcgtcaccgtctcctcg

### CD138 VH Amino Acid Sequence:

qvqlqqsgselmmpgasvkisckatgytfsnywiewvkqrpghglewigeilpgtgrtiynekfkgkatftadissntvqmqlssltsedsavyy carrdyygnfyyamdywgqgtsvtvss (SEQ ID NO: 40)

CDR1-VH: GYTFSNYW (SEQ ID NO: 41)

CDR2-VH: ILPGTGRT (SEQ ID NO: 42)

CDR3-VH: ARRDYYGNFYYAMDY (SEQ ID NO: 43)

CD138 VL nucleotide sequence:
(SEQ ID NO: 44)
gatatccagatgacacagtctacatcctccctgtctgcctctctgggagacagagtcaccatcagttgcagtgcaagtcagggcattaacaa
ttatttaaactggtatcagcagaaaccagatggaactgttgaactcctgatctattacacatcaactttacagtcaggagtcccatcaaggt
tcagtggcagtgggtctgggacagattattctctcaccatcagcaacctggaacctgaagatattggcacttactattgtcagcagtatagt
aagcttcctaggacgttcggtggaggcaccaagctggaaatcaaa

### CD138 VL Amino Acid Sequence:

diqmtqstssisasigdrvtiscsasqginnylnwyqqkpdgtvelliyytstiqsgvpsrfsgsgsgtdysltisnlepedigtyycqqysklprtfgg gtkleik (SEQ ID NO: 45)

CDR1-VL: QGINNY (SEQ ID NO: 46)

CDR2-VL: YTS

CDR3-VL: QQYSKLPRT (SEQ ID NO: 47)

CD319 (CS-1) VH nucleotide sequence:
(SEQ ID NO: 48)
gaggtgcagcttgtcgagtctggaggtggcctggtgcagcctggaggatccctgagactctcctgtgcagcctcaggattcgattttagtaga
tactggatgagttgggtccggcaggctccagggaaagggctagaatggattggagaaattaatccagatagcagtacgataaactatgcgcca
tctctaaaggataaattcatcatctccagagacaacgccaaaaatagcctgtacctgcaaatgaacagtctgagagctgaggacacagccgtt
tattactgtgcaagacctgatgggaactattggtacttcgatgtctggggccagggcaccctggtcaccgtctcctca

### CD319 (CS-1) VH Amino Acid Sequence:

evqlvesggglvqpggslriscaasgfdfsrywmswvrqapgkglewigeinpdsstinyapslkdkfiisrdnaknslylqmnslraedtavyy carpdgnywyfdvwgqgtlvtvss (SEQ ID NO: 49)

CDR1-VH: GFDFSRYW (SEQ ID NO: 50)

CDR2-VH: INPDSSTI (SEQ ID NO: 51)

CDR3-VH: ARPDGNYWYFDV (SEQ ID NO: 52)

CD319 (CS-1) VL nucleotide sequence:
(SEQ ID NO: 53)
gacatccagatgacccagtctccatcctccctgtctgcatctgtaggagacagagtcaccatcacttgcaaggcgagtcaggacgttggcatt
gctgtagcctggtatcagcagaaaccagggaaagttcctaaactcctgatctattgggcatccactcggcacacaggagtcccagatcggttc
agcggcagtggatctgggacagatttcactctcaccatcagcagcctgcagcctgaagatgttgcaacttattactgtcaacagtatagcagt
tacccgtacacttttggccaggggaccaaggtggagatcaaa

### CD319 (CS-1) VL Amino Acid Sequence:

diqmtqspsslsasvgdrvtitckasqdvgiavawyqqkpgkvpklliywastrhtgvpdrfsgsgsgtdftltissiqpedvatyycqqyssypyt fgqgtkveik (SEQ ID NO: 54)

CDR1-VL: QDVGIA (SEQ ID NO: 55)

CDR2-VL: WAS

CDR3-VL: QQYSSYPYT (SEQ ID NO: 56)

### EXAMPLE 10—NKG2D CLONE #3 IS CROSS-REACTIVE WITH MOUSE NKG2D

For the preclinical evaluation cross-species reactivity is beneficial to allow characterization of a potential clinical candidate in preclinical animal models such as mouse models. The NKG2D Clone #3 showed significant binding to mouse and human NKG2D and may therefore represent an interesting candidate for evaluation in mouse models (FIG. 12).

### LITERATURE

1. Carter, P. J. and G. A. Lazar, Next generation antibody drugs: pursuit of the ‘high-hanging fruit’. Nat Rev Drug Discov, 2018. 17(3): p. 197-223.
2. Weng, W. K. and R. Levy, Two immunoglobulin G fragment C receptor polymorphisms independently predict response to rituximab in patients with follicular lymphoma. J Clin Oncol, 2003. 21(21): p. 3940-7.
3. Musolino, A., et al., Immunoglobulin G fragment C receptor polymorphisms and clinical efficacy of trastuzumab-based therapy in patients with HER-2/neu-positive metastatic breast cancer. J Clin Oncol, 2008. 26(11): p. 1789-96.
4. de Haij, S., et al., In vivo cytotoxicity of type I CD20 antibodies critically depends on Fc receptor ITAM signaling. Cancer Res, 2010. 70(8): p. 3209-17.
5. Carter, P. J., Potent antibody therapeutics by design. Nat Rev Immunol, 2006. 6(5): p. 343-57.
6. Kellner, C., et al., Boosting ADCC and CDC activity by Fc engineering and evaluation of antibody effector functions. Methods, 2014. 65(1): p. 105-13.
7. Kohrt, H. E., et al., Combination strategies to enhance antitumor ADCC. Immunotherapy, 2012. 4(5): p. 511-27.
8. Kontermann, R. E. and U. Brinkmann, Bispecific antibodies. Drug Discov Today, 2015. 20(7): p. 838-47.
9. Glorius, P., et al., The novel tribody [(CD20)(2)×CD16] efficiently triggers effector cell-mediated lysis of malignant B cells. Leukemia, 2013. 27(1): p. 190-201.
10. Viardot, A. and R. Bargou, Bispecific antibodies in haematological malignancies. Cancer Treat Rev, 2018. 65: p. 87-95.
11. Bargou, R., et al., Tumor regression in cancer patients by very low doses of a T cell-engaging antibody. Science, 2008. 321(5891): p. 974-7.
12. Topp, M. S., et al., Targeted therapy with the T-cell-engaging antibody blinatumomab of chemotherapy-refractory minimal residual disease in B-lineage acute lymphoblastic leukemia patients results in high response rate and prolonged leukemia-free survival. J Clin Oncol, 2011. 29(18): p. 2493-8.
13. Bauer, S., et al., Activation of NK cells and T cells by NKG2D, a receptor for stress-inducible MICA. Science, 1999. 285(5428): p. 727-9.
14. Lanier, L. L., NKG2D Receptor and Its Ligands in Host Defense. Cancer Immunol Res, 2015. 3(6): p. 575-82.
15. Nausch, N. and A. Cerwenka, NKG2D ligands in tumor immunity. Oncogene, 2008. 27(45): p. 5944-58.
16. Hilpert, J., et al., Comprehensive analysis of NKG2D ligand expression and release in leukemia: implications for NKG2D-mediated NK cell responses. J Immunol, 2012. 189(3): p. 1360-71.
17. Wu, J., et al., An activating immunoreceptor complex formed by NKG2D and DAP10. Science, 1999. 285(5428): p. 730-2.
18. Raulet, D. H., Roles of the NKG2D immunoreceptor and its ligands. Nat Rev Immunol, 2003. 3(10): p. 781-90.
19. Kellner, C., et al., Fusion proteins between ligands for NKG2D and CD20-directed single-chain variable fragments sensitize lymphoma cells for natural killer cell-mediated lysis and enhance antibody-dependent cellular cytotoxicity. Leukemia, 2012. 26(4): p. 830-4.
20. Kellner, C., et al., Enhancing natural killer cell-mediated lysis of lymphoma cells by combining therapeutic antibodies with CD20-specific immunoligands engaging NKG2D or NKp30. Oncoimmunology, 2016. 5(1): p. e1058459.
21. von Strandmann, E. P., et al., A novel bispecific protein (ULBP2-BB4) targeting the NKG2D receptor on natural killer (NK) cells and CD138 activates NK cells and has potent antitumor activity against human multiple myeloma in vitro and in vivo. Blood, 2006. 107(5): p. 1955-62.
22. Chan, W. K., et al., A CS1-NKG2D Bispecific Antibody Collectively Activates Cytolytic Immune Cells against Multiple Myeloma. Cancer Immunol Res, 2018. 6(7): p. 776-787.
23. Ferrari de Andrade, L., et al., Antibody-mediated inhibition of MICA and MICB shedding promotes NK cell-driven tumor immunity. Science, 2018. 359(6383): p. 1537-1542.
24. Groh, V., et al., Costimulation of CD8alphabeta T cells by NKG2D via engagement by MIC induced on virus-infected cells. Nat Immunol, 2001. 2(3): p. 255-60.
25. Maasho, K., et al., NKG2D is a costimulatory receptor for human naive CD8+ T cells. J Immunol, 2005. 174(8): p. 4480-4.
26. Verneris, M. R., et al., Role of NKG2D signaling in the cytotoxicity of activated and expanded CD8+ T cells. Blood, 2004. 103(8): p. 3065-72.
27. Meresse, B., et al., Coordinated induction by IL15 of a TCR-independent NKG2D signaling pathway converts CTL into lymphokine-activated killer cells in celiac disease. Immunity, 2004. 21(3): p. 357-66.
28. Diefenbach, A., et al., Rae1 and H60ligands of the NKG2D receptor stimulate tumour immunity. Nature, 2001. 413(6852): p. 165-71.
29. Hust, M., et al., A human scFv antibody generation pipeline for proteome research. J Biotechnol, 2011. 152(4): p. 159-70.
30. Wirt, T., et al., An Fc Double-Engineered CD20 Antibody with Enhanced Ability to Trigger Complement-Dependent Cytotoxicity and Antibody-Dependent Cell-Mediated Cytotoxicity. Transfus Med Hemother, 2017. 44(5): p. 292-300.
31. Steger, K., et al., CHO-S antibody titers >1 gram/liter using flow electroporation-mediated transient gene expression followed by rapid migration to high-yield stable cell lines. J Biomol Screen, 2015. 20(4): p. 545-51.
32. Oberg, H. H., et al., Novel bispecific antibodies increase gammadelta T-cell cytotoxicity against pancreatic cancer cells. Cancer Res, 2014. 74(5): p. 1349-60.
33. Kellner, C., et al., Mimicking an induced self phenotype by coating lymphomas with the NKp30 ligand B7-H6 promotes NK cell cytotoxicity. J Immunol, 2012. 189(10): p. 5037-46.
34. Chou, T. C. and P. Talalay, Quantitative analysis of dose-effect relationships: the combined effects of multiple drugs or enzyme inhibitors. Adv Enzyme Regul, 1984. 22: p. 27-55.
35. Knappik, A., et al., Fully synthetic human combinatorial antibody libraries (HuCAL) based on modular consensus frameworks and CDRs randomized with trinucleotides. J Mol Biol, 2000. 296(1): p. 57-86.
36. Ewert, S., et al., Biophysical properties of human antibody variable domains. J Mol Biol, 2003. 325(3): p. 531-53.
37. Repp, R., et al., Combined Fc-protein- and Fc-glyco-engineering of scFv-Fc fusion proteins synergistically enhances CD16a binding but does not further enhance NK-cell mediated ADCC. J Immunol Methods, 2011. 373(1-2): p. 67-78.
38. Schewe, D. M., et al., An Fc-engineered CD19 antibody eradicates MRD in patient-derived MLL-rearranged acute lymphoblastic leukemia xenografts. Blood, 2017. 130(13): p. 1543-1552.
39. Peipp, M., et al., CD20-Specific Immunoligands Engaging NKG2D Enhance gammadelta T Cell-Mediated Lysis of Lymphoma Cells. Scand J Immunol, 2017. 86(4): p. 196-206.
40. Inagaki, A., et al., Expression of the ULBP ligands for NKG2D by B-NHL cells plays an important role in determining their susceptibility to rituximab-induced ADCC. Int J Cancer, 2009. 125(1): p. 212-21.
41. Kellner, C., et al., A novel CD19-directed recombinant bispecific antibody derivative with enhanced immune effector functions for human leukemic cells. J Immunother, 2008. 31(9): p. 871-84.
42. Shahied, L. S., et al., Bispecific minibodies targeting HER2/neu and CD16 exhibit improved tumor lysis when placed in a divalent tumor antigen binding format. J Biol Chem, 2004. 279(52): p. 53907-14.
43. Vivier, E., et al., Innate or adaptive immunity? The example of natural killer cells. Science, 2011. 331(6013): p. 44-9.
44. Bryceson, Y. T., et al., Synergy among receptors on resting NK cells for the activation of natural cytotoxicity and cytokine secretion. Blood, 2006. 107(1): p. 159-66.
45. Zalevsky, J., et al., The impact of Fc engineering on an anti-CD19 antibody: increased Fcgamma receptor affinity enhances B-cell clearing in nonhuman primates. Blood, 2009. 113(16): p. 3735-43.
46. Beck, A. and J. M. Reichert, Marketing approval of mogamulizumab: a triumph for glyco-engineering. MAbs, 2012. 4(4): p. 419-25.
47. Lee, H. Z., et al., U.S. Food and drug administration approval: obinutuzumab in combination with chlorambucil for the treatment of previously untreated chronic lymphocytic leukemia. Clin Cancer Res, 2014. 20(15): p. 3902-7.

1. A Fab-scFv fusion protein specifically binding to
(i) an antigen being expressed on the surface of a tumor cell or an autoreactive immune cell via the Fab scaffold, and
(ii) an antigen being expressed on the surface of a leukocyte, preferably cytotoxic lymphocyte via the scFv fragment.

2. The Fab-scFv fusion protein of claim 1, wherein antigen of (ii) is expressed on natural killer (NK) cells, natural killer T (NKT) cells and/or cytotoxic thymocytes (T cells), and/or genetically engineered cells thereof.

3. The Fab-scFv fusion protein of claim 1 or 2, wherein the antigen of (ii) is selected from the group consisting of NKG2D, CD137, NKp30, NKp46, NKp44, 2B4, DNAM-1, CD2, CD4, CD8 and CD28, wherein the antigen is preferably NKG2D.

4. The Fab-scFv fusion protein of claim 1, wherein the antigen of (i) is selected from the group consisting of CD20, CD19, CD22, CD37, CD38, CD7, CD33, CD44, CD54, CD64, CD75s, CD79b, CD96, CD138, CD123, CD317, CD319, BCMA, FCRL5, FLT3, EGFR, HER2, EpCAM CEA, GD2 and Claudin 6/18 wherein the antigen is preferably CD20.

5. The Fab-scFv fusion protein of claim 1, wherein the Fab-scFv fusion protein comprises in case of (i) the six CDRs of SEQ ID NOs 22 to 26 and the CDR2 VL ATS; and/or in case of (ii) the six CDRs of SEQ ID NOs 1 to 5 and the CDR2 VL GNN or SEQ ID NOs 6 to 10 and the CDR2 VL GKN or SEQ ID NOs 11 to 15 and the CDR2 VL GKN.

6. The Fab-scFv fusion protein of claim 1, wherein the Fab-scFv fusion protein comprises
in case of (i) the variable heavy and light chain regions of SEQ ID NOs 27 and 28; and/or
in case of (ii) the variable heavy and light chain regions of SEQ ID NOs 16 and 17 or SEQ ID NOs 18 and 19 or SEQ ID NOs 20 and 21.

7. A nucleic acid sequence or a set of nucleic acid sequences encoding the Fab-scFv fusion protein of claim 1.

8. A vector or a set of vectors encoding the Fab-scFv fusion protein of claim 1 in expressible from.

9. A host cell, preferably a non-human host cell comprising the vector of claim 8.

10. A method for producing the Fab-scFv fusion protein of claim 1 comprising
(a) culturing the host cell of claim 9 under conditions where the host cell expresses the multispecific antibody of claim 1, and
(b) isolating the multispecific antibody of claim 1 as expressed in (a).

11. A pharmaceutical composition comprising the Fab-scFv fusion protein of claim 1, and optionally comprising
(a) an antibody specifically binding to an antigen being expressed on the surface of a tumor cell other than the antigen of (i), wherein the antibody of (a) preferably specifically binds to CD19 or CD38, and/or
(b) an antibody specifically binding to an antigen being expressed on the surface of a cytotoxic lymphocyte other than the antigen of (ii), wherein the antibody of (b) preferably specifically binds to CD3 as expressed on the surface of T cells and NKT cells and/or CD16 or CD32 as expressed on the surface of NK cells, and/or
(c) a cell product, being preferably a chimeric antigen receptor T cell or a chimeric antigen receptor natural killer cell, wherein said cell product expresses the antigen of (ii), and wherein said cell product preferably comprises cytotoxic lymphocytes that are preferably genetically modified to express a synthetic immune receptor containing binding sites to an antigen other than that of (i).

12. The Fab-scFv fusion protein of claim 1, or the pharmaceutical composition of claim 11 for use in treating or preventing a tumor or an autoimmune disease.

13. The Fab-scFv fusion protein, the nucleic acid sequence, the vector, the host cell or the pharmaceutical composition for use of claim 12, wherein in addition
(a) an antibody specifically binding to an antigen being expressed on the surface of a tumor cell other than the antigen of (i) is used, wherein the antibody of (a) preferably specifically binds to CD19 or CD38, and/or
(b) an antibody specifically binding to an antigen being expressed on the surface of a cytotoxic lymphocyte other than the antigen of (ii) is used, wherein the antibody of (b) preferably specifically binds to CD3 as expressed on the surface of T cells or CD16 or CD32 as expressed on the surface of NK cells, and/or
(c) a cell product, being preferably a chimeric antigen receptor T cell or a chimeric antigen receptor natural killer cell, wherein said cell product expresses the antigen of (ii), and wherein said cell product preferably comprises cytotoxic lymphocytes that are preferably genetically modified to express a synthetic immune receptor containing binding sites to an antigen other than that of (i).


SEQUENCE LISTING (56 sequences)

SEQ ID NO: 1 - protein, 8 aa, Artificial Sequence
  Clone #3 CDR1 VH
GYTFTSYG

SEQ ID NO: 2 - protein, 8 aa, Artificial Sequence
  Clone #3 CDR2 VH
IIPIFGTA

SEQ ID NO: 3 - protein, 20 aa, Artificial Sequence
  Clone #3 CDR3 VH
AKDLYDYVWGSYRPNWYFDL

SEQ ID NO: 4 - protein, 6 aa, Artificial Sequence
  Clone #3 CDR1 VL
SLRGYY

SEQ ID NO: 5 - protein, 11 aa, Artificial Sequence
  Clone #3 CDR3 VL
NSRDNNGNFVI

SEQ ID NO: 6 - protein, 8 aa, Artificial Sequence
  Clone #32 CDR1 VH
GFTFSSYG

SEQ ID NO: 7 - protein, 8 aa, Artificial Sequence
  Clone #32 CDR2 VH
ISYDGSNK

SEQ ID NO: 8 - protein, 15 aa, Artificial Sequence
  Clone #32 CDR3 VH
AKDIYDFWSGYIFDY

SEQ ID NO: 9 - protein, 6 aa, Artificial Sequence
  Clone #32 CDR1 VL
SLRSYY

SEQ ID NO: 10 - protein, 13 aa, Artificial Sequence
  Clone #32 CDR3 VL
NSRDSSGNHLDVV

SEQ ID NO: 11 - protein, 8 aa, Artificial Sequence
  Clone #35 CDR1 VH
GFTFDDYT

SEQ ID NO: 12 - protein, 8 aa, Artificial Sequence
  Clone #35 CDR2 VH
ISWNSNKI

SEQ ID NO: 13 - protein, 14 aa, Artificial Sequence
  Clone #35 CDR3 VH
AKDLSAMLSWGIDY

SEQ ID NO: 14 - protein, 6 aa, Artificial Sequence
  Clone #35 CDR1 VL
SLRSYY

SEQ ID NO: 15 - protein, 12 aa, Artificial Sequence
  Clone #35 CDR3 VL
NSRDSSGNHDMV

SEQ ID NO: 16 - protein, 127 aa, Artificial Sequence
  Clone #3 VH
QVQLVQSGAEVKKPGASVKVSCKASGYTFTSYGISWVRQAPGQGLEWMGGIIPIFGTANYAQKFQGRVTITADESTSTAYMELSSLRSEDTAVYYCAKDLYDYVWGSYRPNWYFDLWGRGTLVTVSS

SEQ ID NO: 17 - protein, 108 aa, Artificial Sequence
  Clone #3 VL
SSELTQDPAVSVALGQTVRITCQGDSLRGYYASWYQQKPGQAPVLVTYGNNKRPSGIPDRFSGSSSGNTASLTITGTQAEDEADYYCNSRDNNGNFVIFGGGTKLTVL

SEQ ID NO: 18 - protein, 122 aa, Artificial Sequence
  Clone #32 VH
QVQLQESGGGVVQPGRSLRLSCAASGFTFSSYGMHWVRQAPGKGLEWVAVISYDGSNKYYADSVKGRFTISRDNSKNTLYLQMNSLRGEDTAVYYCAKDIYDFWSGYIFDYWGQGTLVTVSS

SEQ ID NO: 19 - protein, 110 aa, Artificial Sequence
  Clone #32 VL
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHLDVVFGGGTKLTVL

SEQ ID NO: 20 - protein, 121 aa, Artificial Sequence
  Clone #35 VH
QVQLVQSGGVVVQPGGSLRLSCAASGFTFDDYTMHWVRQAPGKGLEWVSGISWNSNKIGYADSVRGRFTISRDNAKNSLFLQMNSLRAEDTALYYCAKDLSAMLSWGIDYWGQGTLVTVSS

SEQ ID NO: 21 - protein, 109 aa, Artificial Sequence
  Clone #35 VL
SSELTQDPAVSVALGQTVRITCQGDSLRSYYASWYQQKPGQAPVLVIYGKNNRPSGIPDRFSGSSSGNTASLTITEAQAEDEADYYCNSRDSSGNHDMVFGGGTKLTVL

SEQ ID NO: 22 - protein, 8 aa, Artificial Sequence
  Rituximab CDR1 VH
GYTFTSYN

SEQ ID NO: 23 - protein, 8 aa, Artificial Sequence
  Rituximab CDR2 VH
IYPGNGDT

SEQ ID NO: 24 - protein, 14 aa, Artificial Sequence
  Rituximab CDR3 VH
ARSTYYGGDWYFNV

SEQ ID NO: 25 - protein, 5 aa, Artificial Sequence
  Rituximab CDR1 VL
SSVSY

SEQ ID NO: 26 - protein, 9 aa, Artificial Sequence
  Rituximab CDR3 VL
QQWTSNPPT

SEQ ID NO: 27 - protein, 121 aa, Artificial Sequence
  Rituximab VH
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSS

SEQ ID NO: 28 - protein, 106 aa, Artificial Sequence [sequence omitted]

SEQ ID NO: 29 - protein, 18 aa, Artificial Sequence
  Linker
GSASAPKLEEGEFSEARV

SEQ ID NO: 30 - DNA, 360 bp, Artificial Sequence
  4D5 (Trastuzumab) VH
gaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactattggggtcaaggaaccctggtcaccgtctcctcg

SEQ ID NO: 31 - protein, 120 aa, Artificial Sequence [sequence omitted]

SEQ ID NO: 32 - protein, 4 aa, Artificial Sequence
  4D5 (Trastuzumab) VH CDR1
GFNI

SEQ ID NO: 33 - protein, 8 aa, Artificial Sequence
  4D5 (Trastuzumab) VH CDR2
IYPTNGYT

SEQ ID NO: 34 - protein, 13 aa, Artificial Sequence
  4D5 (Trastuzumab) VH CDR3
SRWGGDGFYAMDY

SEQ ID NO: 35 - DNA, 321 bp, Artificial Sequence
  4D5 (Trastuzumab) VL
gatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctattctggagtcccttctcgcttctctggatccagatctgggacggatttcactctgaccatcagcagtctgcagccggaagacttcgcaacttattactgtcagcaacattatactactcctcccacgttcggacagggtaccaaggtggagatcaaa

SEQ ID NO: 36 - protein, 107 aa, Artificial Sequence [sequence omitted]

SEQ ID NO: 37 - protein, 6 aa, Artificial Sequence
  4D5 (Trastuzumab) VL CDR1
QDVNTA

SEQ ID NO: 38 - protein, 9 aa, Artificial Sequence
  4D5 (Trastuzumab) VL CDR3
QQHYTTPPT

SEQ ID NO: 39 - DNA, 366 bp, Artificial Sequence
  CD138 VH
caggtgcagctgcagcagtctggatccgagctgatgatgcctggggcctcagtgaagatatcctgcaaggctactggctacacattcagtaactactggatagagtgggtaaagcagaggcctggacatggccttgagtggattggagagattttacctggaacaggtaggactatatacaatgagaagttcaagggcaaggccacattcactgcagatatttcctccaacacagtccagatgcaactcagcagcctgacatctgaggactctgccgtctattactgtgcaagaagggactattacggcaacttctactatgctatggactactggggccaagggaccagcgtcaccgtctcctcg

SEQ ID NO: 40 - protein, 122 aa, Artificial Sequence
  CD138 VH
QVQLQQSGSELMMPGASVKISCKATGYTFSNYWIEWVKQRPGHGLEWIGEILPGTGRTIYNEKFKGKATFTADISSNTVQMQLSSLTSEDSAVYYCARRDYYGNFYYAMDYWGQGTSVTVSS

SEQ ID NO: 41 - protein, 8 aa, Artificial Sequence
  CD138 VH CDR1
GYTFSNYW

SEQ ID NO: 42 - protein, 8 aa, Artificial Sequence
  CD138 VH CDR2
ILPGTGRT

SEQ ID NO: 43 - protein, 15 aa, Artificial Sequence
  CD138 VH CDR3
ARRDYYGNFYYAMDY

SEQ ID NO: 44 - DNA, 321 bp, Artificial Sequence
  CD138 VL
gatatccagatgacacagtctacatcctccctgtctgcctctctgggagacagagtcaccatcagttgcagtgcaagtcagggcattaacaattatttaaactggtatcagcagaaaccagatggaactgttgaactcctgatctattacacatcaactttacagtcaggagtcccatcaaggttcagtggcagtgggtctgggacagattattctctcaccatcagcaacctggaacctgaagatattggcacttactattgtcagcagtatagtaagcttcctaggacgttcggtggaggcaccaagctggaaatcaaa

SEQ ID NO: 45 - protein, 107 aa, Artificial Sequence
  CD138 VL
DIQMTQSTSSLSASLGDRVTISCSASQGINNYLNWYQQKPDGTVELLIYYTSTLQSGVPSRFSGSGSGTDYSLTISNLEPEDIGTYYCQQYSKLPRTFGGGTKLEIK

SEQ ID NO: 46 - protein, 6 aa, Artificial Sequence
  CD138 VL CDR1
QGINNY

SEQ ID NO: 47 - protein, 9 aa, Artificial Sequence
  CD138 VL CDR3
QQYSKLPRT

SEQ ID NO: 48 - DNA, 357 bp, Artificial Sequence
  CD319 (CS-1) VH
gaggtgcagcttgtcgagtctggaggtggcctggtgcagcctggaggatccctgagactctcctgtgcagcctcaggattcgattttagtagatactggatgagttgggtccggcaggctccagggaaagggctagaatggattggagaaattaatccagatagcagtacgataaactatgcgccatctctaaaggataaattcatcatctccagagacaacgccaaaaatagcctgtacctgcaaatgaacagtctgagagctgaggacacagccgtttattactgtgcaagacctgatgggaactattggtacttcgatgtctggggccagggcaccctggtcaccgtctcctca

SEQ ID NO: 49 - protein, 119 aa, Artificial Sequence
  CD319 (CS-1) VH
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 50 - protein, 8 aa, Artificial Sequence
  CD319 (CS-1) VH CDR1
GFDFSRYW

SEQ ID NO: 51 - protein, 8 aa, Artificial Sequence
  CD319 (CS-1) VH CDR2
INPDSSTI

SEQ ID NO: 52 - protein, 12 aa, Artificial Sequence
  CD319 (CS-1) VH CDR3
ARPDGNYWYFDV

SEQ ID NO: 53 - DNA, 321 bp, Artificial Sequence
  CD319 (CS-1) VL
gacatccagatgacccagtctccatcctccctgtctgcatctgtaggagacagagtcaccatcacttgcaaggcgagtcaggacgttggcattgctgtagcctggtatcagcagaaaccagggaaagttcctaaactcctgatctattgggcatccactcggcacacaggagtcccagatcggttcagcggcagtggatctgggacagatttcactctcaccatcagcagcctgcagcctgaagatgttgcaacttattactgtcaacagtatagcagttacccgtacacttttggccaggggaccaaggtggagatcaaa

SEQ ID NO: 54 - protein, 107 aa, Artificial Sequence
  CD319 (CS-1) VL
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIK

SEQ ID NO: 55 - protein, 6 aa, Artificial Sequence
  CD319 (CS-1) VL CDR1
QDVGIA

SEQ ID NO: 56 - protein, 9 aa, Artificial Sequence
  CD319 (CS-1) VL CDR3
QQYSSYPYT